(19) United States
(12) Patent Application Publication
(10) Pub. No.: US 2024/0245705 A1
(43) Pub. Date: Jul. 25, 2024

(54) ORALLY ADMINISTERED COMBINATIONS OF AMOXICILLIN AND AVIBACTAM DERIVATIVES FOR TREATING MYCOBACTERIAL INFECTIONS

(71) Applicant: Arixa Pharmaceuticals, Inc., New York, NY (US)

(72) Inventor: JOAQUIM TRIAS, Millbrae, CA (US)

(73) Assignee: Arixa Pharmaceuticals, Inc., New York, NY (US)

(21) Appl. No.: 18/618,149

(22) Filed: Mar. 27, 2024

Related U.S. Application Data
(63) Continuation of application No. 17/123,858, filed on Dec. 16, 2020, now abandoned.
(60) Provisional application No. 62/951,187, filed on Dec. 20, 2019.

(51) Int. Cl. A61K 31/551; A61K 9/00; A61K 31/43

(52) U.S. Cl. CPC A61K 31/551; A61K 9/0053; A61K 31/43

(57) ABSTRACT

Pharmaceutical compositions comprise amoxicillin and an avibactam derivative and methods of treating bacterial infections using the pharmaceutical compositions. The pharmaceutical compositions can be formulated for oral administration and following oral administration provide a therapeutically effective amount of amoxicillin and avibactam in the plasma of a patient. The oral pharmaceutical compositions and methods can be used to treat mycobacterial infections.

### FIELD

The present disclosure relates to orally administered combinations of amoxicillin and avibactam derivatives. The pharmaceutical compositions can be used to treat mycobacterial infections.

### BACKGROUND

Resistance to commonly used β-lactam anti-infectives is related to expression of β-lactamases by the targeted bacteria. β-Lactamase enzymes can hydrolyze the β-lactam ring of β-lactam antibiotics, thus rendering the β-lactam antibiotics ineffective against the β-lactamase-producing bacteria. Inhibition of β-lactamases by a suitable substrate can prevent degradation of the β-lactam antibiotic, thereby increasing the effectiveness of the administered β-lactam antibiotic and mitigating the emergence of resistance.

Avibactam is a β-lactamase inhibitor approved for intravenous use in combination with ceftazidime, a cephalosporin antibiotic, to treat intraabdominal infections, urinary tract infections and pneumonia. Avibactam derivatives that provide therapeutically effective plasma concentrations of avibactam when administered orally have been developed. When co-administered with amoxicillin, the avibactam derivatives provide the opportunity to treat bacterial infections caused by bacteria producing β-lactamase enzymes with oral administration.

Mycobacteria are naturally resistant to most β-lactams because the presence of β-lactamases and the permeability barrier of the cell wall. Story-Roller, et al., Front Microbiol. 2018 9:2273. A limited number of IV β-lactams are used for therapy of non-tuberculous mycobacteria NTM). Floto, et al., Thorax. 2016 71:88-90. For example, cefoxitin or imipenem are used for the treatment of infections caused by M. abscessus because these antibiotics are stable to hydrolysis by mycobacterial β-lactamases. Therapy using β-lactam antibiotics is lengthy, with a recommended initial treatment phase of up to 12 weeks and requires an intravenous (IV) infusion several times per day. A maintenance phase of treatment can extend to one year or longer. Floto et al., Id. There are no oral β-lactam antibiotics available to treat NTM because none of the orally available β-lactam antibiotics are sufficiently potent. For example, the minimal inhibitory concentration (MIC) of NTM to amoxicillin, an IV oral antibiotic, is often well above the concentrations reached at the site of the infection after oral administration, which precludes clearing the infection. An effective oral treatment could be used not only during the initial treatment phase for NTM infections, but also during the maintenance phase (approximately 12 months), where only oral or inhaled antibiotics are more conveniently used.

### SUMMARY

According to the present invention, pharmaceutical compositions comprise: amoxicillin or a pharmaceutically acceptable salt thereof; and an avibactam derivative of Formula (1):

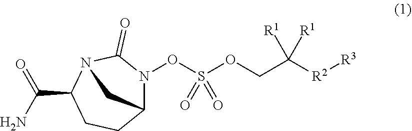

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

According to the present invention, oral dosage forms comprise the pharmaceutical composition according to the present invention.

According to the present invention, kits comprise a pharmaceutical composition according to the present invention.

According to the present invention, kits comprise: a first pharmaceutical composition comprising amoxicillin or a pharmaceutically acceptable salt thereof; and a second pharmaceutical composition comprising an avibactam derivative of Formula (1):

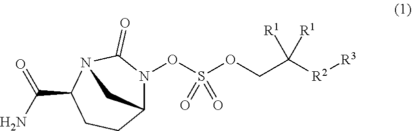

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
   R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

According to the present invention, methods of treating a bacterial infection in a patient in need of such treatment comprise orally administering to the patient: a therapeutically effective amount of amoxicillin or a pharmaceutically acceptable salt thereof; and a therapeutically effective amount of an avibactam derivative of Formula (1):

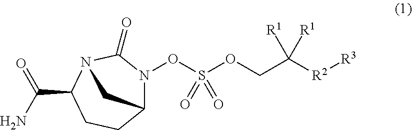

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
   R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

According to the present invention, methods of treating a bacterial infection in a patient in need of such treatment comprise orally administering to the patient a therapeutically effective amount of a pharmaceutical composition according to the present invention,

### DETAILED DESCRIPTION

A dash (“-”) that is not between two letters or symbols is used to indicate a point of attachment for a moiety or substituent. For example, —CONH₂ is attached through the carbon atom.

“Alkyl” refers to a saturated, branched, or straight-chain, monovalent hydrocarbon radical derived by the removal of one hydrogen atom from a single carbon atom of a parent alkane, alkene, or alkyne. An alkyl group can be, for example, C₁₋₁₀ alkyl, C₁₋₆ alkyl, C₁₋₅ alkyl, C₁₋₄ alkyl, or C₁₋₃ alkyl. An alkyl can be, for example, methyl, ethyl, n-propyl, iso-propyl, n-butyl, tert-butyl or iso-butyl.

“Alkoxy” refers to a radical —OR where R is alkyl as defined herein. Examples of alkoxy groups include methoxy, ethoxy, propoxy, and butoxy. An alkoxy group can be C₁₋₆ alkoxy, C₁₋₅ alkoxy, C₁₋₄ alkoxy, C₁₋₃ alkoxy, ethoxy, or methoxy.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms selected from N, O, and S. For such fused, bicyclic ring systems wherein only one of the rings is a carbocyclic aromatic ring, the radical carbon atom may be at the carbocyclic aromatic ring or at the heterocycloalkyl ring. Examples of aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene, and the like. An aryl group can be C₆₋₁₀ aryl, C₆₋₉ aryl, C₆₋₈ aryl, or phenyl. Aryl, however, does not encompass or overlap in any way with heteroaryl, separately defined herein.

“Arylalkyl” refers to an acyclic alkyl radical in which one of the hydrogen atoms bonded to a carbon atom is replaced with an aryl group. Examples of arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, and 2-naphthophenylethan-1-yl. Where specific alkyl moieties are intended, the nomenclature arylalkanyl, arylalkenyl, or arylalkynyl is used. An arylalkyl group can be C₇₋₁₆ arylalkyl, e.g., the alkanyl, alkenyl or alkynyl moiety of the arylalkyl group is C₁₋₆ and the aryl moiety is C₆₋₁₀ An arylalkyl group can be C₇₋₁₆ arylalkyl, such as the alkanyl, alkenyl or alkynyl moiety of the arylalkyl group is C₁₋₆ and the aryl moiety is C₆₋₁₀. An arylalkyl group can be C₇₋₉ arylalkyl, wherein the alkyl moiety can be C₁₋₃ alkyl and the aryl moiety can be phenyl. An arylalkyl group can be C₇₋₁₆ arylalkyl, C₇₋₁₄ arylalkyl, C₇₋₁₂ arylalkyl, C₇₋₁₀ arylalkyl, C₇₋₈ arylalkyl, or benzyl.

“Avibactam derivative” refers to an avibactam derivative of Formula (1), a pharmaceutically acceptable salt thereof, a hydrate thereof, a solvate thereof, or a combination of any of the forgoing. An avibactam derivative of Formula (1) includes sub-genuses and specific compounds within the scope of Formula (1). When orally administered, an avibactam derivative provides avibactam in the plasma of a patient.

“Avibactam equivalents” refers to the amount of avibactam in an avibactam derivative provided the by the present disclosure. Avibactam derivatives provided by the present disclosure are absorbed within the gastrointestinal tract and release avibactam in the systemic circulation. The avibactam derivatives comprise a promoiety that enhances absorption of avibactam from the gastrointestinal tract. Avibactam has a molecular weight of 265.25 Da, and the corresponding avibactam derivative will have a greater molecular weight due to the promoiety. For example, the avibactam derivative ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate has a molecular weight of 393.41 Da. Thus, this avibactam derivative comprises 0.674 avibactam equivalents. When orally administered, assuming 100% bioavailability and 100% in vivo conversion efficiency, 1 mg of the avibactam derivative will provide 0.674 mg avibactam in the systemic circulation of a patient. The avibactam equivalents provided by a particular avibactam derivative will depend, at least in part, on factors affecting the oral bioavailability of the particular avibactam derivative such as, for example, the stability of the avibactam derivative in the gastrointestinal tract, the extent of absorption into the systemic circulation, and the conversion efficiency of the avibactam derivative to avibactam in the systemic circulation. The percent oral bioavailability accounts for these multiple factors. Avibactam derivatives provided by the present disclosure can exhibit an oral bioavailability in a patient such as a human, for example, greater than 20% F, greater than 30%, greater than 40% F, greater than 50% F, or greater than 60% F. For example, a 1 mg dose of the avibactam derivative ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate having an oral bioavailability, for example, of 25% can provide 0.25 mg avibactam in the systemic circulation of a patient.

“Bioavailability” refers to the rate and amount of a drug that reaches the systemic circulation of a patient following administration of the drug or prodrug thereof to the patient and can be determined by evaluating, for example, the plasma concentration-versus-time profile for a drug. Parameters useful in characterizing a plasma or blood concentration-versus-time curve include the area under the curve (AUC), the time to maximum concentration (Tₘₐₓ), and the maximum drug concentration (Cₘₐₓ), where Cₘₐₓ is the maximum concentration of a drug in the plasma of a patient following administration of a dose of the drug or form of drug to the patient, and Tₘₐₓ is the time to the maximum concentration (Cₘₐₓ) of a drug in the plasma of a patient following administration of a dose of the drug or form of drug to the patient.

“Oral bioavailability” (F %) refers to the fraction of an orally administered drug that reaches systemic circulation compared to a comparable dose delivered intravenously.

“Compounds” and moieties provided by the present disclosure include any specific compounds within these formulae. Compounds may be identified either by their chemical structure and/or chemical name. Compounds are named using the ChemBioDraw Professional Version 17.1.0.105 (19) (CambridgeSoft, Cambridge, MA) nomenclature/structure program. When the chemical structure and chemical name conflict, the chemical structure is determinative of the identity of the compound. The compounds described herein may comprise one or more stereogenic centers and/or double bonds and therefore may exist as stereoisomers such as double-bond isomers (i.e., geometric isomers), enantiomers, diastereomers, or atropisomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds and moieties provided by the present disclosure include optical isomers of compounds and moieties, racemates thereof, and other mixtures thereof. In such embodiments, the single enantiomers or diastereomers may be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column with chiral stationary phases. In addition, compounds include (Z)- and (E)-forms (or cis- and trans-forms) of compounds with double bonds either as single geometric isomers or mixtures thereof.

Compounds and moieties may also exist in several tautomeric forms including the enol form, the keto form, and mixtures thereof. Accordingly, the chemical structures depicted herein encompass all possible tautomeric forms of the illustrated compounds. Compounds may exist in unsolvated forms as well as solvated forms, including hydrated forms. Certain compounds may exist in multiple crystalline, co-crystalline, or amorphous forms. Compounds include pharmaceutically acceptable salts thereof, or a pharmaceutically acceptable solvates of the free acid form of any of the foregoing, as well as crystalline forms of any of the foregoing

“Cycloalkyl” refers to a saturated or partially unsaturated cyclic alkyl radical. A cycloalkyl group can be C₃₋₆ cycloalkyl, C₃₋₅ cycloalkyl, C₅₋₆ cycloalkyl, cyclopropyl, cyclopentyl, or cyclohexyl. A cycloalkyl can be selected from, for example, cyclopropyl, cyclobutyl, cyclopentyl, and cyclohexyl.

“Cycloalkylalkyl” refers to an acyclic alkyl radical in which one of the hydrogen atoms bonded to a carbon atom is replaced with a cycloalkyl group as defined herein. A cycloalkylalkyl group can be C₄₋₃₀ cycloalkylalkyl, for example, the alkyl moiety of the cycloalkylalkyl group is C₁₋₁₀ and the cycloalkyl moiety of the cycloalkylalkyl moiety is C₃₋₂₀. A cycloalkylalkyl group can be C₄₋₂₀ cycloalkylalkyl for example, the alkanyl, alkenyl, or alkynyl moiety of the cycloalkylalkyl group is C₁₋₈ and the cycloalkyl moiety of the cycloalkylalkyl group is C₃₋₁₂. A cycloalkylalkyl can be C₄₋₉ cycloalkylalkyl, wherein the alkyl moiety of the cycloalkylalkyl group is C₁₋₃ alkyl, and the cycloalkyl moiety of the cycloalkylalkyl group is C₃₋₆ cycloalkyl. A cycloalkylalkyl group can be C₄₋₁₂ cycloalkylalkyl, C₄₋₁₀ cycloalkylalkyl, C₄₋₈ cycloalkylalkyl, and C₄₋₆ cycloalkylalkyl. A cycloalkylalkyl group can be cyclopropylmethyl (—CH₂-cyclo-C₃H₅), cyclopentylmethyl (—CH₂-cyclo-C₅H₉), or cyclohexylmethyl (—CH₂-cyclo-C₆H₁₁). A cycloalkylalkyl group can be cyclopropylethenyl (—CH═CH-cyclo-C₃H₅), or cyclopentylethynyl (—C≡C-cyclo-C₅H₉).

“Cycloalkylheteroalkyl” by itself or as part of another substituent refers to a heteroalkyl group in which one or more of the carbon atoms (and certain associated hydrogen atoms) of an alkyl group are independently replaced with the same or different heteroatomic group or groups and in which one of the hydrogen atoms bonded to a carbon atom is replaced with a cycloalkyl group. In a cycloalkylheteroalkyl, the heteroatomic group can be selected from —O—, —S—, —NH—, —N(—CH₃)—, —SO—, and —SO₂—, or the heteroatomic group can be selected from —O— and —NH—, or the heteroatomic group is —O— or —NH—.

“Cycloalkyloxy” refers to a radical —OR where R is cycloalkyl as defined herein. Examples of cycloalkyloxy groups include cyclopropyloxy, cyclobutyloxy, cyclopentyloxy, and cyclohexyloxy. A cycloalkyloxy group can be C₃₋₆ cycloalkyloxy, C₃₋₅ cycloalkyloxy, C₅₋₆ cycloalkyloxy, cyclopropyloxy, cyclobutyloxy, cyclopentyloxy, or cyclohexyloxy.

“Disease” refers to a disease, disorder, condition, or symptom of any of the foregoing.

“Heteroalkoxy” refers to an alkoxy group in which one or more of the carbon atoms are replaced with a heteroatom. A heteroalkoxy group can be, for example, C₁₋₆ heteroalkoxy, C₁₋₅ heteroalkoxy, C₁₋₄ heteroalkoxy, or C₁₋₃ heteroalkoxy. In a heteroalkoxy, the heteroatomic group can be selected from —O—, —S—, —NH—, —NR—, —SO₂—, and —SO₂—, or the heteroatomic group can be selected from —O— and —NH—, or the heteroatomic group is —O— and —NH—. A heteroalkoxy group can be C₁₋₆ heteroalkoxy, C₁₋₅ heteroalkoxy, C₁₋₄ heteroalkoxy, or C₁₋₃ heteroalkoxy.

“Heteroalkyl” by itself or as part of another substituent refer to an alkyl group in which one or more of the carbon atoms (and certain associated hydrogen atoms) are independently replaced with the same or different heteroatomic group or groups. Examples of heteroatomic groups include —O—, —S—, —NH—, —NR—, —O—O—, —S—S—, ═N—N═, —N═N—, —N═N—NR—, —PR—, —P(O)OR—, —P(O)R—, —POR—, —SO—, —SO₂—, —Sn(R)₂—, and the like, where each R can independently be selected from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, and substituted C₇₋₁₈ heteroarylalkyl. Each R in a heteroatomic group can be independently selected from hydrogen and C₁₋₃ alkyl. Reference to, for example, a C₁₋₆ heteroalkyl, means a C₁₋₆ alkyl group in which at least one of the carbon atoms (and certain associated hydrogen atoms) is replaced with a heteroatom. For example, C₁₋₆ heteroalkyl includes groups having five carbon atoms and one heteroatom, groups having four carbon atoms and two heteroatoms, and so forth. In a heteroalkyl, the heteroatomic group can be selected from —O—, —S—, —NH—, —N(—CH₃)—, —SO—, and —SO₂—, or the heteroatomic group can be selected from —O— and —NH—, or the heteroatomic group can be —O— or —NH—. A heteroalkyl group can be C₁₋₆ heteroalkyl, C₁₋₅ heteroalkyl, or C₁₋₄ heteroalkyl, or C₁₋₃ heteroalkyl.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which may be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. When the total number of N, S, and O atoms in the heteroaryl group exceeds one, the heteroatoms may or may not be adjacent to one another. The total number of heteroatoms in the heteroaryl group is not more than two. In a heteroaryl, the heteroatomic group can be selected from —O—, —S—, —NH—, —N(—CH₃)—, —S(O)—, and —SO₂—, or the heteroatomic group can be selected from —O— and —NH—, or the heteroatomic group can be —O— or —NH—. A heteroaryl group can be selected from, for example, C₅₋₁₀ heteroaryl, C₅₋₉ heteroaryl, C₅₋₈ heteroaryl, C₅₋₇ heteroaryl, C₅₋₆ heteroaryl, C₅ heteroaryl, or C₆ heteroaryl.

Examples of suitable heteroaryl groups include groups derived from acridine, arsindole, carbazole, α-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, or oxazolidine. A heteroaryl group can be derived from thiophene, pyrrole, benzothiophene, benzofuran, indole, pyridine, quinoline, imidazole, oxazole, or pyrazine. For example, a heteroaryl can be C₅ heteroaryl and can be selected from furyl, thienyl, pyrrolyl, imidazolyl, pyrazolyl, isothiazolyl, or isoxazolyl. A heteroaryl can be C₆ heteroaryl, and can be selected from pyridinyl, pyrazinyl, pyrimidinyl, and pyridazinyl.

“Heteroarylalkyl” refers to an arylalkyl group in which one of the carbon atoms (and certain associated hydrogen atoms) is replaced with a heteroatom. A heteroarylalkyl group can be, for example, C₆₋₁₆ heteroarylalkyl, C₆₋₁₄ heteroarylalkyl, C₆₋₁₂ heteroarylalkyl, C₆₋₁₀ heteroarylalkyl, C₆₋₈ heteroarylalkyl, C₇ heteroarylalkyl, or C₆ heteroarylalkyl. In a heteroarylalkyl, the heteroatomic group can be selected from, for example, —O—, —S—, —NH—, —N(—CH₃)—, —SO—, and —SO₂—, or the heteroatomic group can be selected from —O— and —NH—, or the heteroatomic group can be —O— or —NH—.

“Heterocycloalkyl” by itself or as part of another substituent refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system violates the Hückel-rule. Examples of heteroatoms to replace the carbon atom(s) include N, P, O, S, and Si. Examples of heterocycloalkyl groups include groups derived from epoxides, azirines, thiiranes, imidazolidine, morpholine, piperazine, piperidine, pyrazolidine, pyrrolidine, and quinuclidine. A heterocycloalkyl can be C₅ heterocycloalkyl and can be selected from pyrrolidinyl, tetrahydrofuranyl, tetrahydrothiophenyl, imidazolidinyl, oxazolidinyl, thiazolidinyl, doxolanyl, and dithiolanyl. A heterocycloalkyl can be C₆ heterocycloalkyl and can be selected from piperidinyl, tetrahydropyranyl, piperizinyl, oxazinyl, dithianyl, and dioxanyl. A heterocycloalkyl group can be C₃₋₆ heterocycloalkyl, C₃₋₅ heterocycloalkyl, C₅₋₆ heterocycloalkyl, C₅ heterocycloalkyl or C₆ heterocycloalkyl. In a heterocycloalkyl, the heteroatomic group can be selected from —O—, —S—, —NH—, —N(—CH₃)—, —SO—, and —SO₂—, or the heteroatomic group can be selected from —O— and —NH—, or the heteroatomic group can be —O— or —NH—.

“Heterocycloalkylalkyl” refers to a cycloalkylalkyl group in which one or more carbon atoms (and certain associated hydrogen atoms) of the cycloalkyl ring are independently replaced with the same or different heteroatom. A heterocycloalkylalkyl can be, for example, C₄₋₁₂ heterocycloalkylalkyl, C₄₋₁₀ heterocycloalkylalkyl, C₄₋₈ heterocycloalkylalkyl, C₄₋₆ heterocycloalkylalkyl, C₆₋₇ heterocycloalkylalkyl, or C₆ heterocycloalkylalkyl or C₇ heterocycloalkylalkyl. In a heterocycloalkylalkyl, the heteroatomic group can be selected from —O—, —S—, —NH—, —N(—CH₃)—, —SO—, and —SO₂—, or the heteroatomic group can be selected from —O— and —NH—, or the heteroatomic group can be —O— or —NH—.

“Parent aromatic ring system” refers to an unsaturated cyclic or polycyclic ring system having a cyclic conjugated π (pi) electron system with 4n+2 electrons (Hückel rule). Included within the definition of “parent aromatic ring system” are fused ring systems in which one or more of the rings are aromatic and one or more of the rings are saturated or unsaturated, such as, for example, fluorene, indane, indene, or phenalene. Examples of parent aromatic ring systems include aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, and trinaphthalene.

“Hydrate” refers to a compound in which water is incorporated into the crystal lattice, in a stoichiometric proportion, resulting in the formation of an adduct. Methods of making hydrates include, for example, storage in an atmosphere containing water vapor, dosage forms that include water, or routine pharmaceutical processing steps such as, for example, crystallization such as from water or mixed aqueous solvents, lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. A compound can be, for example, a monohydrate, a dihydrate, or a trihydrate.

“Metabolic intermediate” refers to a compound that is formed in vivo by metabolism of a parent compound and that further undergoes reaction in vivo to release an active agent. Compounds of Formula (1) are protected sulfonate nucleophile prodrugs of the non-β-lactam β-lactamase inhibitor avibactam that are metabolized in vivo to provide avibactam ([2S,5R]-2-carbamoyl-7-oxo-1,6-diazabicyclo[3.2.1]octan-6-yl hydrogen sulfate). Metabolic intermediates undergo nucleophilic cyclization to release avibactam and one or more reaction products. It is desirable that the reaction products or metabolites not be toxic.

“Neopentyl” refers to a radical in which a methylene carbon is bonded to a carbon atom, which is bonded to three non-hydrogen substituents. Examples of non-hydrogen substituents include carbon, oxygen, nitrogen, and sulfur. Each of the three non-hydrogen substituents can be carbon. Two of the three non-hydrogen substituents can be carbon, and the third non-hydrogen substituent can be selected from oxygen and nitrogen. A neopentyl group can have the structure:

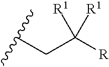

where each R¹ and R can be defined as for Formula (1).

“Parent aromatic ring system” refers to an unsaturated cyclic or polycyclic ring system having a conjugated π electron system. Included within the definition of “parent aromatic ring system” are fused ring systems in which one or more of the rings are aromatic and one or more of the rings are saturated or unsaturated, such as, for example, fluorene, indane, indene, and phenalene. Examples of parent aromatic ring systems include aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of π-electrons corresponding to the Hückel rule (4n+2). Examples of heteroatoms to replace the carbon atoms include N, P, O, S, and Si. Included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, and xanthene. Examples of parent heteroaromatic ring systems include arsindole, carbazole, R-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, and oxazolidine.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids and one or more protonable functional groups such as primary, secondary, or tertiary amines within the parent compound. Examples of inorganic acids include hydrochloric acid, hydrobromic acid, sulfuric acid, nitric acid, and phosphoric acid. A salt can be formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. A salt can be formed when one or more acidic protons present in the parent compound are replaced by a metal ion, such as an alkali metal ion, an alkaline earth ion, or an aluminum ion, or combinations thereof; or coordinates with an organic base such as ethanolamine, diethanolamine, triethanolamine, and N-methylglucamine. A pharmaceutically acceptable salt can be a hydrochloride salt. A pharmaceutically acceptable salt can be a sodium salt. In compounds having two or more ionizable groups, a pharmaceutically acceptable salt can comprise one or more counterions, such as a bi-salt, for example, a dihydrochloride salt.

The term “pharmaceutically acceptable salt” includes hydrates and other solvates, as well as salts in crystalline or non-crystalline form. Where a particular pharmaceutically acceptable salt is disclosed, it is understood that the particular salt such as a hydrochloride salt, is an example of a salt, and that other salts may be formed using techniques known to one of skill in the art. Additionally, one of skill in the art would be able to convert the pharmaceutically acceptable salt to the corresponding compound, free base and/or free acid, using techniques generally known in the art. A pharmaceutically acceptable salt can include pharmaceutically acceptable esters.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to amoxicillin or a pharmaceutically acceptable salt thereof and/or an avibactam derivative of Formula (1) or a pharmaceutically acceptable salt thereof and at least one pharmaceutically acceptable vehicle, with which amoxicillin or a pharmaceutically acceptable salt thereof and/or an avibactam derivative of Formula (1) or a pharmaceutically acceptable salt thereof is administered to a patient.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease “Preventing” or “prevention” refers to reducing symptoms of the disease by taking the compound in a preventative fashion. The application of a therapeutic for preventing or prevention of a disease of disorder is known as prophylaxis.

“Prodrug” refers to a derivative of a drug molecule that requires a transformation within the body to release the active drug. Prodrugs are frequently, although not necessarily, pharmacologically inactive until converted to the parent drug. Avibactam derivatives of Formula (1) are prodrugs of avibactam.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example, following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature or pH.

The agent may be endogenous to the conditions of use, such as an enzyme present in the systemic circulation of a patient to which the prodrug is administered or the acidic conditions of the stomach or the agent may be supplied exogenously. For example, for an avibactam derivative of Formula (1), the promoiety can have the structure:

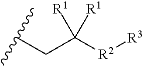

where R¹, R², and R³ are defined as for Formula (1).

“Single bond” as in the expression “R² is selected from a single bond” refers to a moiety in which R² is a single bond (—). For example, in a moiety having the structure —C(R¹)₂—R²—R³, where R² is a single bond, —R²— corresponds to a single bond, “—”, and the moiety has the structure —C(R¹)₂—R³.

“Solvate” refers to a molecular complex of a compound with one or more solvent molecules in a stoichiometric or non-stoichiometric amount. Such solvent molecules are those commonly used in the pharmaceutical arts, which are known to be innocuous to a patient, such as water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water. Methods of making solvates include, for example, storage in an atmosphere containing a solvent, dosage forms that include the solvent, or routine pharmaceutical processing steps such as, for example, crystallization (i.e., from solvent or mixed solvents) vapor diffusion. Solvates may also be formed, under certain circumstances, from other crystalline solvates or hydrates upon exposure to the solvent or upon suspension material in solvent. Solvates may crystallize in more than one form resulting in solvate polymorphism.

“Substituted” refers to a group in which one or more hydrogen atoms are independently replaced with the same or different substituent(s). Each substituent can be independently selected from deuterio, halogen, —OH, —CN, —CF₃, —OCF₃, ═O, —NO₂, C₁₋₆ alkoxy, C₁₋₆ alkyl, —COOR, —NR₂, and —CONR₂; wherein each R is independently selected from hydrogen and C₁₋₆ alkyl. Each substituent can be independently selected from deuterio, halogen, —NH₂, —OH, C₁₋₃ alkoxy, and C₁₋₃ alkyl, trifluoromethoxy, and trifluoromethyl. Each substituent can be independently selected from, for example, deuterio, —OH, methyl, ethyl, trifluoromethyl, methoxy, ethoxy, and trifluoromethoxy. Each substituent can be selected from, for example, deuterio, C₁₋₃ alkyl, ═O, C₁₋₃ alkyl, C₁₋₃ alkoxy, and phenyl. Each substituent can be selected from, for example, deuterio, —OH, —NH₂, C₁₋₃ alkyl, and C₁₋₃ alkoxy.

“Treating” or “treatment” of a disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to alleviating one or more symptoms resulting from the disease, diminishing the extent of the disease, stabilizing the disease such as preventing or delaying the worsening of the disease, preventing or delaying the spread of the disease, preventing or delaying the recurrence of the disease, delaying or slowing the progression of the disease, ameliorating the disease state, providing a remission, either partial or total, of the disease, decreasing the dose of one or more other medications required to treat the disease, delaying the progression of the disease, increasing the quality of life, and/or prolonging survival.

“Treating” or “treatment” also refers to inhibiting the disease, either physically, (e.g., stabilization of a discernible symptom), physiologically, (e.g., stabilization of a physical parameter), or both, and to inhibiting at least one physical parameter or manifestation that may or may not be discernible to the patient. “Treating” or “treatment” also refers to delaying the onset of the disease or at least one or more symptoms thereof in a patient who may be exposed to or predisposed to a disease or disorder even though that patient does not yet experience or display symptoms of the disease.

“Therapeutically effective amount” refers to the amount of a compound that, when administered to a patient for treating a disease, or at least one of the clinical symptoms of a disease, is sufficient to affect such treatment of the disease or symptom thereof. A “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Therapeutically effective amount” means the amount of a compound that, when administered to a patient for treating a disease, is sufficient to treat the disease. The “therapeutically effective amount” will vary depending, for example, on the compound, the disease and its severity and the age, weight, adsorption, distribution, metabolism and excretion, of the patient to be treated. In reference to a bacterial infection, a therapeutically effective amount can comprise an amount sufficient to cause the total number of bacteria present in a patient to diminish and/or to slow the growth rate of the bacteria. A therapeutically effective amount can be an amount sufficient to prevent or delay recurrence of the bacterial infection. A therapeutically effective amount can reduce the number of bacterial cells; inhibit, retard, slow to some extent and preferably stop bacterial cell proliferation; prevent or delay occurrence and/or recurrence of the bacterial infection; and/or relieve to some extent one or more of the symptoms associated with the bacterial infection.

“Simultaneous administration,” means that a first administration and a second administration in a combination therapy are done within a time separation of less than 30 minutes, such as less than 15 minutes, less than 10 minutes, less than 5 minutes, or less than 1 minute. For example, two therapeutically active compounds can be simultaneously administered in a single dosage form or in two separate dosage forms.

“Sequential administration” means that a first administration and a second administration are administered within a time separation, for example, of greater than 30 minutes, greater than 60 minutes or greater than 120 minutes. For example, two therapeutically active compounds can be sequentially administered in two separate dosage forms.

“Vehicle” refers to a diluent, excipient or carrier with which a compound is administered to a patient. In some embodiments, the vehicle is pharmaceutically acceptable.

“MIC” refers to the minimum inhibitory concentration of an antimicrobial agent that will inhibit the growth, such as the visible growth, of a microorganism after a certain time of incubation, for example, after overnight incubation. MIC₉₀ and MIC₅₀ are metrics used to assess the in vitro susceptibility of a cohort of bacterial isolates to a specific antimicrobial agents or combination of antimicrobial agents using the testing method. MIC₉₀ and MIC₅₀ values refer to the lowest concentration of the antibiotic at which 90% and 50% of the isolates are inhibited, respectively. A MIC₉₀ can be defined as the lowest concentration of an antibiotic at which the growth of 90% of microorganism isolates are inhibited after overnight incubation. A MIC₅₀ can be defined as the lowest concentration of an antibiotic at which the growth of 50% of microorganism isolates are inhibited after overnight, such as after a 12 hour, incubation.

“Pharmacokinetics” (PK) refers to the time course of drug concentrations in plasma resulting from a particular dosing regimen.

“Pharmacodynamics” (PD) refers to the relationship between drug concentrations in plasma a resulting pharmacological effect.

“The PK/PD Index” for an antimicrobial agent is a parameter of pharmacodynamics expressed as bacteriostasis, 1-log kill or 2-log kill, and is associated with the pharmacokinetics to constitute an exposure-response relationship (PK/PD) that is adjusted for the MIC of a given bacterial isolate. The most common PK/PD measures associated with efficacy are the area under the concentration-time curve (AUC) to MIC ratio (AUC:MIC), peak concentration (Cₘₐₓ) to MIC ratio (Cₘₐₓ:MIC), the percentage of time that drug concentrations exceed the MIC over the dosing interval (T>MIC), and the percentage of time that drug concentrations exceed a concentration threshold (T>Ct). To reflect free or unbound or microbiologically active drug, the PK/PD indices can be corrected for plasma protein binding and expressed as fAUC:MIC, fCₘₐₓ:MIC, fT>MIC and fT>Cₜ.

Reference is now made in detail to certain embodiments of compounds, compositions, and methods. The disclosed embodiments are not intended to be limiting of the claims. To the contrary, the claims are intended to cover all alternatives, modifications, and equivalents.

Pharmaceutical compositions provided by the disclosure comprise amoxicillin and an avibactam derivative that when orally administered provide a therapeutically effective amount of amoxicillin and avibactam in the plasma of a patient for treating a bacterial infection such as a mycobacterial infection.

Methods provided by the present disclosure include methods of treating a mycobacterial infection in a patient comprising orally administering to a patient in need of such treatment a therapeutically effective amount of amoxicillin or a pharmaceutically acceptable salt thereof and an avibactam derivative or a pharmaceutically acceptable salt thereof.

The susceptibility of mycobacteria such as M. abscessus or M. ulcerans to amoxicillin is very low in the absence of a β-lactamase inhibitor. Dubee et al., J Antimicrob Chemother. 2015 70:1051-8; Arenaz-Callao et al., PLoS Negl Trop Dis. 2019. 13:e0007126. However, avibactam potentiates amoxicillin and lowers MICs using discovery in vitro methods to concentrations that are 8 μg/mL to 16 μg/mL or lower. These susceptibility methods have not been optimized and modifications in the method could lead to even lower MIC values. The efficacy of amoxicillin and other β-lactam antibiotics is driven by the time of free drug >MIC. Usually the percent of time above MIC is required to be from about 30% to 6% depending of the bacterial species and the β-lactam antibiotic. de Velde et al. shows that dosing regimens of amoxicillin at concentrations from 500 mg to 1000 mg can achieve sustained blood levels above a MIC of 8 μg/mL between dosing intervals. de Velde, J Antimicrob Chemother. 2016. 71:2909-17. Susceptibility breakpoint for amoxicillin when tested against other non-mycobacterial pathogens is 8 μg/mL, and 16 μg/mL for intermediate strains. Clinical and Laboratory Standards Institute (CLSI), 2019. Performance Standards for Antimicrobial Susceptibility Testing—Twenty-Ninth Informational Supplement. CLSI Document M100S; PA, USA.

These findings suggest that a treatment based on oral amoxicillin and oral avibactam prodrug is possible. This treatment should deliver sustained levels of amoxicillin over time to clear an infection by strains with MICs 8 μg/mL to 16 μg/mL or lower (an in vitro method to determine MIC has not been developed yet, so values could be even lower). This dosing regimen may require frequent oral dosing up to Q6 h. This range of MICs is similar to that used for cefoxitin or imipenem when treating mycobacterial infections and suggests that if the MICs of amoxicillin are similar, it should be possible to design an oral amoxicillin treatment regimen that in combination with oral delivery of avibactam with a prodrug can be effective in treating mycobacterial infections. Although the value of the % free concentration that is associated with efficacy is not known for mycobacterial infections and amoxicillin, one can predict that for MICs that are in the range of from 8 μg/mL to 16 μg/mL or lower, a dosing regimen of amoxicillin at 500 mg, 875 mg, or 1000 mg, administered q6 h, q8 h or q12 h, could lead to efficacy and clear an infection. Because the MICs of amoxicillin in mycobacteria can be much higher (Dubee et al., J Antimicrob Chemother. 2015 70:1051-8), the oral delivery of avibactam should effectively increase the potency of orally administered amoxicillin. Amoxicillin is well-suited for long term therapy based on the safety of the drug.

Amoxicillin is in the class of β-lactam antibiotics. Amoxicillin, (1S,4S,7S)-7-((R)-2-amino-2-(4-hydroxyphenyl)acetamido)-3,3-dimethyl-2-thia-6-azabicyclo[3.2.0]heptane-4-carboxylic acid, has the structure:

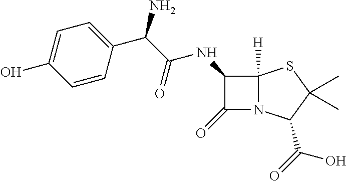 [SMILES: CC1(C)S[C@@H]2[C@H](NC(=O)[C@H](N)c3ccc(O)cc3)C(=O)N2[C@H]1C(=O)O]

β-lactams act by binding to penicillin-binding proteins that inhibit a process called transpeptidation, leading to activation of autolytic enzymes in the bacterial cell wall. This process leads to lysis of the cell wall, and thus, the destruction of the bacterial cell. This type of activity is referred to as bactericidal killing.

Amoxicillin is used to treat infections caused by Gram-positive bacteria and Gram-negative bacteria including most Streptococcus species including Listeria monocytogenes, Enterococcus, Haemophilus influenzae, some Escherichia coli, Actinomyces, Clostridial species, Salmonella, Shigella, and Corynebacteria.

Amoxicillin is FDA approved for the treatment of genitourinary tract infections, ear, nose, and throat infections, lower respiratory tract infections, Helicobacter pylori infections, pharyngitis, tonsillitis, and skin and skin structure infections. Amoxicillin is recommended as the first-line treatment by the Infectious Disease Society of America (IDSA), for acute bacterial rhinosinusitis and as one of the treatments for community-acquired pneumonia.

Amoxicillin can be administered in combination with a β-lactamase inhibitor such as clavulanic acid and sulbactam. These β-lactamase inhibitors work by binding irreversibly to the catalytic site of an organism's penicillinase enzyme, which causes resistance to the original beta-lactam ring. These drugs when combined with amoxicillin, β-lactamase inhibitors can broaden spectrum amoxicillin to organisms that produce the penicillinase enzyme.

Avibactam derivatives provided by the present disclosure are sulfonate ester prodrugs of the non-β-lactam β-lactamase inhibitor, avibactam ([2S,5R]-2-carbamoyl-7-oxo-1,6-diazabicyclo[3.2.1]octan-6-yl hydrogen sulfate; (1R,2R,5R)-2-carbamoyl-7-oxo-1,6-diazabicyclo[3.2.1]octan-6-yl hydrogen sulfate (ChemDraw Professional 17.1.0.105 (19)), which has the structure:

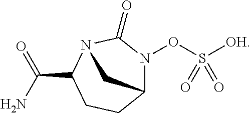

Avibactam derivatives that provide a bioavailability of avibactam in the systemic circulation of a patient following oral administration are disclosed in U.S. Pat. No. 10,085,999.

In the avibactam prodrugs a nucleophilic moiety is positioned proximate to the hydrogen sulfate group. In vivo, the nucleophilic moiety reacts to release avibactam. Avibactam is an inhibitor of class A, class C, and certain Class D β-lactamases and can be useful in the treatment of bacterial infections when used in combination with amoxicillin.

Avibactam derivatives can have the structure of Formula (1):

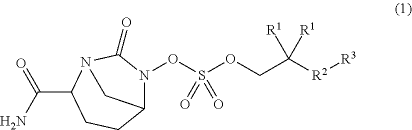

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₁₀ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
   R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl. Compounds of Formula (1) can have the stereochemistry of Formula (1a):

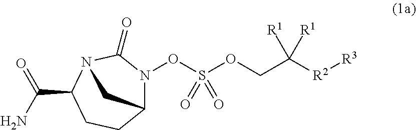

In compounds of Formula (1), each R¹ can independently be C₁₋₆ alkyl.

In compounds of Formula (1), each R¹ can independently be methyl, ethyl, or n-propyl.

In compounds of Formula (1), each R¹ can be same and is methyl, ethyl, or n-propyl.

In compounds of Formula (1), each R¹ is methyl.

In compounds of Formula (1), each R¹ together with the geminal carbon atom to which they are bonded can form a C₃₋₆ cycloalkyl ring or a substituted C₃₋₆ cycloalkyl ring.

In compounds of Formula (1), each R¹ together with the geminal carbon atom to which they are bonded can form a C₃₋₆ cycloalkyl ring. For example, each R¹ together with the geminal carbon atom to which they are bonded can form a cyclopropyl ring, a cyclobutyl ring, a cyclopentyl ring, or a cyclohexyl ring.

In compounds of Formula (1), each R¹ each R¹ together with the geminal carbon atom to which they are bonded can form a C₃₋₆ heterocycloalkyl ring or a substituted C₃₋₆ heterocycloalkyl ring.

In compounds of Formula (1), R² can be selected from a single bond, C₁₋₂ alkanediyl, and substituted C₁₋₂ alkanediyl.

In compounds of Formula (1), R² can be a single bond.

In compounds of Formula (1), R² can be a single bond; and R³ can be C₁₋₆ alkyl.

In compounds of Formula (1), R² can be selected from C₁₋₂ alkanediyl and substituted C₁₋₂ alkanediyl.

In compounds of Formula (1), R² can be methanediyl, ethanediyl, substituted methanediyl, or substituted ethanediyl.

In compounds of Formula (1), R² can be substituted C₁₋₂ alkanediyl where the substituent group can be selected from —OH, —CN, —CF₃, —OCF₃, ═O, —NO₂, C₁₋₆ alkoxy, C₁₋₆ alkyl, —COOR, —NR₂, and —CONR₂; wherein each R is independently selected from hydrogen and C₁₋₆ alkyl.

In compounds of Formula (1), R² can be substituted C₁₋₂ alkanediyl where the substituent group can be a nucleophilic group. For example, R² can be substituted C₁₋₂ alkanediyl where the substituent group can be selected from —OH, —CF₃, —O—CF₃, —NO₂, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), where each R⁴ is defined as for Formula (1), or each R⁴ is selected from hydrogen and C₁₋₈ alkyl.

In compounds of Formula (1), R² can be substituted C₁₋₂ alkanediyl where the substituent group is selected from —OH, —O—C(O)—R⁴, —S—C(O)—R⁴, —NH—C(O)—R⁴, —C(O)—O—R⁴, —C(O)—S—R⁴, —C(O)—NH—R⁴, —S—S—R⁴, —S—R⁴, —NH—R⁴, —CH(—NH₂)(—R⁴), substituted C₅₋₆ aryl, —NHR⁴, —CH(—NH₂)(—R⁴); and R⁴ is defined as for Formula (1), or each R⁴ is selected from hydrogen and C₁₋₈ alkyl.

In compounds of Formula (1), where R² is substituted C₁₋₆ alkanediyl, substituted C₁₋₆ heteroalkanediyl, or substituted C₅₋₆ arenediyl, the stereochemistry of the carbon atom to which the substituent group is bonded can be of the (S) configuration.

In compounds of Formula (1), where R² is substituted C₁₋₆ alkanediyl, substituted C₁₋₆ heteroalkanediyl, or substituted C₅-6 arenediyl, the stereochemistry of the carbon atom to which the substituent group is bonded can be of the (R) configuration.

In compounds of Formula (1), R² is selected from C₅₋₆ cycloalkanediyl, C₅₋₆ heterocycloalkanediyl, C₅₋₆ arenediyl, and C₅₋₆ heterocycloalkanediyl.

In compounds of Formula (1), R² can be cyclopenta-1,3-diene-diyl, substituted cyclopenta-1,3-diene-diyl, benzene-diyl or substituted benzene-diyl. For example, R² can be 1,2-benzene-diyl or substituted 1,2-benzene-diyl.

In compounds of Formula (1), R³ can be selected from —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴); where R⁴ is defined as for Formula (1), or each R⁴ is selected from hydrogen and C₁₋₈ alkyl.

In compounds of Formula (1), R³ can be selected from —O—C(O)—R⁴, —C(O)—O—R⁴, —S—C(O)—R⁴, —C(O)—S—R⁴, —S—S—R⁴, —NH—R⁴, and —CH(—NH₂)(—R⁴); where R⁴ is defined as for Formula (1), or each R⁴ is selected from hydrogen and C₁₋₈ alkyl.

In compounds of Formula (1), R³ is —C(O)—O—R⁴); where R⁴ is defined as for Formula (1), or each R⁴ is selected from hydrogen and C₁₋₈ alkyl.

In compounds of Formula (1), R⁴ can be selected from hydrogen, C₁₋₃ alkyl, C₅₋₆ cycloalkyl, C₅₋₆ heterocycloalkyl, C₅₋₆ aryl, substituted C₁₋₃ alkyl, substituted C₅₋₆ cycloalkyl, substituted C₅₋₆ heterocycloalkyl, and substituted C₅₋₆ aryl.

In compounds of Formula (1), R⁴ can be selected from methyl, ethyl, phenyl, and benzyl.

In compounds of Formula (1), R⁴ can be selected from hydrogen and C₁₋₈ alkyl.

In compounds of Formula (1), R⁴ can be selected from C₁₋₈ alkyl, C₁₋₈ heteroalkyl, C₇₋₉ arylalkyl, C₅₋₇ heterocycloalkyl, substituted C₁₋₈ alkyl, substituted C₁₋₈ heteroalkyl, substituted C₇₋₉ arylalkyl, and substituted C₅₋₇ heterocycloalkyl.

In compounds of Formula (1), R⁴ can be selected from C₁₋₈ alkyl, C₁₋₈ heteroalkyl, C₇₋₉ arylalkyl, and C₅₋₇ heterocycloalkyl.

In compounds of Formula (1), R⁴ can be selected from methyl, ethyl, n-propyl, isopropyl, n-butyl, sec-butyl isobutyl, tert-butyl, 2-methoxyethyl, methylbenzene, oxetane-3-oxy-yl, cyclopentyl, cyclohexyl, and 2-pyrrolidinyl.

In compounds of Formula (1), R³ can be —C(O)—O—R⁴; and R⁴ can be selected from C₁₋₈ alkyl, C₁₋₈ heteroalkyl, C₅₋₇ cycloalkyl, C₅₋₇ heterocycloalkyl, C₆ aryl, C₇₋₉ arylalkyl, substituted C₁₋₈ alkyl, substituted C₁₋₈ heteroalkyl, substituted C₅₋₆ cycloalkyl, substituted C₅₋₆ heterocycloalkyl, substituted C₆ aryl, and C₇₋₉ arylalkyl.

In compounds of Formula (1), R³ can be —C(O)—O—R⁴; and R⁴ can be selected from C₁₋₈ alkyl, C₁₋₈ heteroalkyl, C₇₋₉ arylalkyl, C₅₋₇ heterocycloalkyl, substituted C₁₋₈ alkyl, substituted C₁₋₈ heteroalkyl, substituted C₇₋₉ arylalkyl, and substituted C₅₋₇ heterocycloalkyl.

In compounds of Formula (1), R³ can be —C(O)—O—R⁴; and R⁴ can be selected from C₁₋₈ alkyl, C₁₋₈ heteroalkyl, C₇₋₉ arylalkyl, and C₅₋₇ heterocycloalkyl.

In compounds of Formula (1), R³ can be selected from —O—C(O)—CH₃, —O—C(O)—CH₂—CH₃, —O—C(O)-phenyl, —O—C(O)—CH₂-phenyl, —S—C(O)—CH₃, —S—C(O)—CH₂—CH₃, —S—C(O)— phenyl, —S—C(O)—CH₂-phenyl, —NH—C(O)—CH₃, —NH—C(O)—CH₂—CH₃, —NH—C(O)-phenyl, —NH—C(O)—CH₂-phenyl, —O—C(O)—O—CH₃, —O—C(O)—O—CH₂—CH₃, —O—C(O)—O-phenyl, —O—C(O)—O—CH₂-phenyl, —S—C(O)—O—CH₃, —S—C(O)—O—CH₂—CH₃, —S—C(O)—O-phenyl, —S—C(O)—O—CH₂-phenyl, —NH—C(O)—O—CH₃, —NH—C(O)—O—CH₂—CH₃, —NH—C(O)—O-phenyl, —NH—C(O)—O—CH₂-phenyl, —C(O)—O—CH₃, —C(O)—O—CH₂—CH₃, —C(O)—O-phenyl, —C(O)—O—CH₂-phenyl, —C(O)—S—CH₃, —C(O)—S—CH₂—CH₃, —C(O)—S-phenyl, —C(O)—S—CH₂-phenyl, —C(O)—NH—CH₃, —C(O)—NH—CH₂—CH₃, —C(O)—NH-phenyl, —C(O)—NH—CH₂-phenyl, —O—C(O)—O—CH₃, —O—C(O)—O—CH₂—CH₃, —O—C(O)—O-phenyl, —O—C(O)—O—CH₂-phenyl, —O—C(O)—S—CH₃, —O—C(O)—S—CH₂—CH₃, —O—C(O)—S-phenyl, —O—C(O)—S—CH₂-phenyl, —O—C(O)—NH—CH₃, —O—C(O)—NH—CH₂—CH₃, —O—C(O)—NH-phenyl, —O—C(O)—NH—CH₂-phenyl, —S—SH, —S—S—CH₃, —S—S—CH₂—CH₃, —S—S-phenyl, —S—S—CH₂-phenyl, —SH, —S—CH₃, —S—CH₂—CH₃, —S— phenyl, —S—CH₂-phenyl, —NH₂, —NH—CH₃, —NH—CH₂—CH₃, —NH-phenyl, —NH—CH₂-phenyl, —CH(—NH₂)(—CH₃), —CH(—NH₂)(—CH₂—CH₃), —CH(—NH₂)(-phenyl), and —CH(—NH₂)(—CH₂-phenyl).

In compounds of Formula (1), R³ can be selected from C₅₋₆ cycloalkyl, C₅₋₆ heterocycloalkyl, C₅₋₆ aryl, C₅₋₆ heteroaryl, substituted C₅₋₆ cycloalkyl, substituted C₅₋₆ heterocycloalkyl, substituted C₅₋₆ aryl, and substituted C₅₋₆ heteroaryl, comprising at least one nucleophilic group. For example, R³ can have the structure of Formula (2a) or Formula (2b):

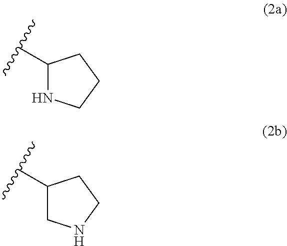 [SMILES: *C1CCCN1 ; *C1CCNC1]

In compounds of Formula (1), R⁴ can be selected from C₁₋₃ alkyl, C₅₋₆ cycloalkyl, C₅₋₆ heterocycloalkyl, C₅₋₆ aryl, substituted C₁₋₃ alkyl, substituted C₅₋₆ cycloalkyl, substituted C₅₋₆ heterocycloalkyl, and substituted C₅₋₆ aryl.

In compounds of Formula (1), each R¹ together with the carbon atom to which they are bonded form a C₄₋₆ heterocycloalkyl ring comprising two adjacent S atoms or a substituted C₄₋₆ heterocycloalkyl ring comprising at least one heteroatom selected from O and S, and a carbonyl (═O) substituent group bonded to a carbon atom adjacent the at least one heteroatom.

In compounds of Formula (1), R² can be a bond; R³ can be C₁₋₃ alkyl; and each R¹ together with the carbon atom to which they are bonded form a C₄₋₆ heterocycloalkyl ring comprising two adjacent S atoms or a substituted C₄₋₆ heterocycloalkyl ring comprising at least one heteroatom selected from O and S, and a ═O substituent group bonded to a carbon atom adjacent the heteroatom.

In compounds of Formula (1), the promoiety —CH₂—C(R¹)₂—R³—R⁴ can have any of the following structures, where R³ can be C₁₋₆ alkyl, such as C₁₋₄ alkyl, such as methyl or ethyl:

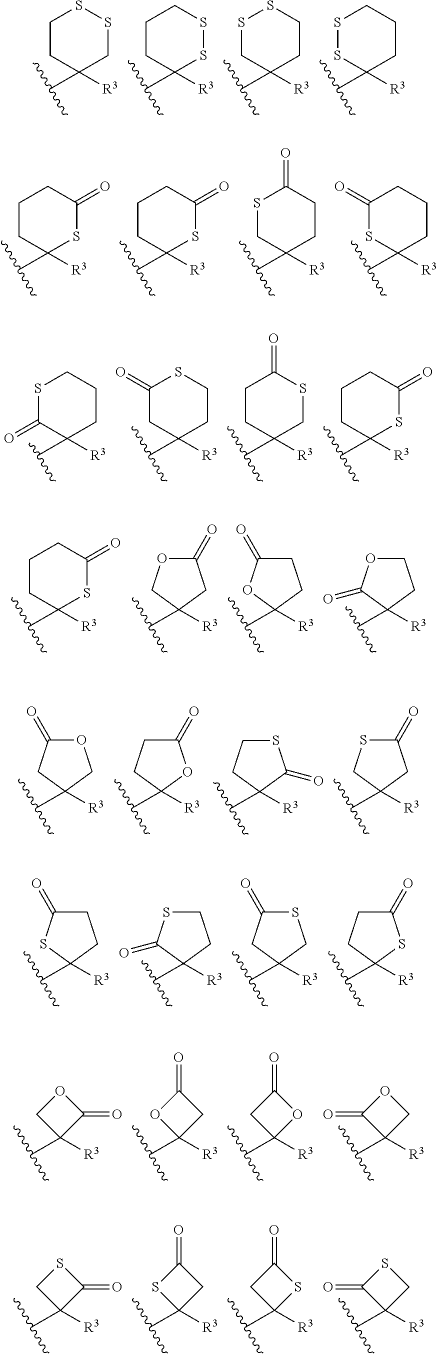

In compounds of Formula (1), R² can be a single bond; R³ can be C₁₋₃ alkyl; and each R¹ together with the carbon atom to which they are bonded can form a C₄₋₆ heterocycloalkyl ring or a substituted C₄₋₆ heterocycloalkyl ring.

In compounds of Formula (1), R² can be a single bond; R³ can be C₁₋₃ alkyl; and each R¹ together with the carbon atom to which they are bonded can form a C₄₋₆ heterocycloalkyl ring comprising two adjacent S atoms or a substituted C₄₋₆ heterocycloalkyl ring comprising at least one heteroatom selected from O and S, and a carbonyl (═O) substituent group bonded to a carbon atom adjacent the heteroatom.

In compounds of Formula (1), R² can be a single bond; R³ can be C₁₋₃ alkyl; and each R¹ together with the carbon atom to which they are bonded can form a 1,2-dithiolane, 1,2-diethane ring, thietan-2-one ring, dihydrothiophen-2(3H)-one ring, tetrahydro-2H-thiopyran-2-one ring, oxetan-2-one ring dihydrofuran-2(3H)-one ring, or tetrahydro-2H-pyran-2-one ring.

In compounds of Formula (1),
each R¹ can be methyl;
R² can be selected from a single bond, methanediyl, ethanediyl, —CH(—OH)—, —CH(—O—C(O)—CH₂CH₃)—, and 1,2-benzene-diyl; and
R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴), where R⁴ can be selected from hydrogen, methyl, ethyl, cyclopentyl, cyclohexyl, phenyl, benzyl, and 2-pyrrolidinyl.

In compounds of Formula (1),
each R¹ and the geminal carbon to which they are bonded can form a C₃₋₆ cycloalkyl ring;
R² can be selected from a bond, methanediyl, ethanediyl, —CH(—OH)—, —CH(—O—C(O)—CH₂CH₃)—, and 1,2-benzene-diyl; and
R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴), where R⁴ can be selected from hydrogen, methyl, ethyl, cyclopentyl, cyclohexyl, phenyl, benzyl, and 2-pyrrolidinyl.

In compounds of Formula (1),
R² can be a bond;
R³ be C₁₋₃ alkyl; and
each R¹ together with the carbon atom to which they are bonded can form a 1,2-dithiolante, 1,2-dithane ring, thietan-2-one ring, dihydrothiophen-2(3H)-one ring, tetrahydro-2H-thiopyran-2-one ring, oxetan-2-one ring dihydrofuran-2(3H)-one ring, or tetrahydro-2H-pyran-2-one ring.

In compounds of Formula (1), each R¹ can be methyl;
R² can be selected from a single bond, methanediyl, ethanediyl, —CH(—OH)—, —CH(—O—C(O)—CH₂CH₃)—, and 1,2-benzene-diyl; and
R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴);
wherein R⁴ can be selected from C₁₋₈ alkyl, C₁₋₈ heteroalkyl, C₇₋₉ arylalkyl, and C₅₋₇ heterocycloalkyl.

In compounds of Formula (1),
each R¹ can be methyl;
R² can be selected from a single bond, methanediyl, ethanediyl, —CH(—OH)—, —CH(—O—C(O)—CH₂CH₃)—, and 1,2-benzene-diyl; and
R³ can be —C(O)—O—R⁴;
wherein R⁴ can be selected from C₁₋₈ alkyl, C₁₋₈ heteroalkyl, C₇₋₉ arylalkyl, and C₅₋₇ heterocycloalkyl.

In compounds of Formula (1),
each R¹ can be methyl;
R² can be selected from a single bond, methanediyl, ethanediyl, —CH(—OH)—, —CH(—O—C(O)—CH₂CH₃)—, and 1,2-benzene-diyl; and
R³ can be selected from —O—C(O)—R⁴, —C(O)—O—R⁴, —S—C(O)—R⁴, —C(O)—S—R⁴, —S—S—R⁴, —NHR⁴, and —CH(—NH₂)(—R⁴);
wherein R⁴ can be selected from methyl, ethyl, n-propyl, isopropyl, n-butyl, sec-butyl isobutyl, tert-butyl, 2-methoxyethyl, methylbenzene, oxetane-3-oxy-yl, cyclopentyl, cyclohexyl, and 2-pyrrolidinyl.

In compounds of Formula (1),
each R¹ can be methyl;
R² can be selected from a single bond, methanediyl, ethanediyl, —CH(—OH)—, —CH(—O—C(O)—CH₂CH₃)—, and 1,2-benzene-diyl; and
R³ can be —C(O)—O—R⁴;
wherein R⁴ can be selected from methyl, ethyl, n-propyl, isopropyl, n-butyl, sec-butyl isobutyl, tert-butyl, 2-methoxyethyl, methylbenzene, oxetane-3-oxy-yl, cyclopentyl, cyclohexyl, and 2-pyrrolidinyl.

In compounds of Formula (1),
each R¹ can be methyl;
R² can be a single bond; and
R³ can be —C(O)—O—R⁴;
wherein R⁴ can be selected from C₁₋₁₀ alkyl, C₁₋₁₀ heteroalkyl, C₇₋₁₀ alkylarene, and C₅₋₁₀ heterocycloalkylalkyl.

In compounds of Formula (1),
each R¹ can be methyl;
R² can be a single bond;
R³ can be —C(O)—O—R⁴, wherein R⁴ can be selected from C₁₋₁₀ alkyl, C₁₋₁₀ heteroalkyl, C₇₋₁₀ alkylarene, and C₅₋₁₀ heterocycloalkylalkyl; and
each of R⁵, R⁶, and R⁷ can be hydrogen.

In compounds of Formula (1), the compound can be selected from:
3-(((((1R,2S,5R)-2-carbamoyl-7-oxo-1,6-diazabicyclo[3.2.1]octan-6-yl)oxy)sulfonyl)oxy)-2,2-dimethylpropyl benzoate (2);
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3);
benzyl 3-(((((1R,2S,5R)-2-carbamoyl-7-oxo-1,6-diazabicyclo[3.2.1]octan-6-yl)oxy)sulfonyl)oxy)-2,2-dimethylpropanoate (4);
4-(((((1R,2S,5R)-2-carbamoyl-7-oxo-1,6-diazabicyclo[3.2.1]octan-6-yl)oxy)sulfonyl)oxy)-3,3-dimethylbutyl benzoate (6);
4-(((((1R,2S,5R)-2-carbamoyl-7-oxo-1,6-diazabicyclo[3.2.1]octan-6-yl)oxy)sulfonyl)oxy)-3,3-dimethylbutyl propionate (7);
benzyl (4-(((((1R,2S,5R)-2-carbamoyl-7-oxo-1,6-diazabicyclo[3.2.1]octan-6-yl)oxy)sulfonyl)oxy)-3,3-dimethylbutyl) adipate (8);
6-(4-(((((1R,2S,5R)-2-carbamoyl-7-oxo-1,6-diazabicyclo[3.2.1]octan-6-yl)oxy)sulfonyl)oxy)-3,3-dimethylbutoxy)-6-oxohexanoic acid (9);
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (10);
isopropyl 3-(((((1R,2S,5R)-2-carbamoyl-7-oxo-1,6-diazabicyclo[3.2.1]octan-6-yl)oxy)sulfonyl)oxy)-2,2-dimethylpropanoate (11);
hexyl 3-(((((1R,2S,5R)-2-carbamoyl-7-oxo-1,6-diazabicyclo[3.2.1]octan-6-yl)oxy)sulfonyl)oxy)-2,2-dimethylpropanoate (12);
heptyl 3-(((((1R,2S,5R)-2-carbamoyl-7-oxo-1,6-diazabicyclo[3.2.1]octan-6-yl)oxy)sulfonyl)oxy)-2,2-dimethylpropanoate (13);
tert-butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (14);
2-methoxyethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (15);
oxetan-3-yl 3-(((((1R,2S,5R)-2-carbamoyl-7-oxo-1,6-diazabicyclo[3.2.1]octan-6-yl)oxy)sulfonyl)oxy)-2,2-dimethylpropanoate (16);
ethyl 1-((((((1R,2S,5R)-2-carbamoyl-7-oxo-1,6-diazabicyclo[3.2.1]octan-6-yl)oxy)sulfonyl)oxy)methyl)cyclohexanecarboxylate (17);
ethyl 1-((((((1R,2S,5R)-2-carbamoyl-7-oxo-1,6-diazabicyclo[3.2.1]octan-6-yl)oxy)sulfonyl)oxy)methyl)cyclopropanecarboxylate (18);
ethyl 1-((((((1R,2S,5R)-2-carbamoyl-7-oxo-1,6-diazabicyclo[3.2.1]octan-6-yl)oxy)sulfonyl)oxy)methyl)cyclobutanecarboxylate (19);
(1R,2S,5R)-2-carbamoyl-7-oxo-1,6-diazabicyclo[3.2.1]octan-6-yl 1H-imidazole-1-sulfonate (34);
ethyl 5-(((((1R,2S,5R)-2-carbamoyl-7-oxo-1,6-diazabicyclo[3.2.1]octan-6-yl)oxy)sulfonyl)oxy)-4,4-dimethylpentanoate (35);
hexyl 5-(((((1R,2S,5R)-2-carbamoyl-7-oxo-1,6-diazabicyclo[3.2.1]octan-6-yl)oxy)sulfonyl)oxy)-4,4-dimethylpentanoate (36);
heptyl 5-(((((1R,2S,5R)-2-carbamoyl-7-oxo-1,6-diazabicyclo[3.2.1]octan-6-yl)oxy)sulfonyl)oxy)-4,4-dimethylpentanoate (37);
2-methoxyethyl 5-(((((1R,2S,5R)-2-carbamoyl-7-oxo-1,6-diazabicyclo[3.2.1]octan-6-yl)oxy)sulfonyl)oxy)-4,4-dimethylpentanoate (38);
5-(((((1R,2S,5R)-2-carbamoyl-7-oxo-1,6-diazabicyclo[3.2.1]octan-6-yl)oxy)sulfonyl)oxy)-2,2,4,4-tetramethylpentyl propionate (39);
5-(((((1R,2S,5R)-2-carbamoyl-7-oxo-1,6-diazabicyclo[3.2.1]octan-6-yl)oxy)sulfonyl)oxy)-2,2,4,4-tetramethylpentyl benzoate (40);
5-(((((1R,2S,5R)-2-carbamoyl-7-oxo-1,6-diazabicyclo[3.2.1]octan-6-yl)oxy)sulfonyl)oxy)-2,2,4,4-tetramethylpentyl 2,6-dimethylbenzoate (41);
(1R,2S,5R)-2-carbamoyl-7-oxo-1,6-diazabicyclo[3.2.1]octan-6-yl ((3-methyl-2-oxotetrahydrofuran-3-yl)methyl) sulfate (42);
3-(((((1R,2S,5R)-2-carbamoyl-7-oxo-1,6-diazabicyclo[3.2.1]octan-6-yl)oxy)sulfonyl)oxy)-2,2-dimethylpropyl pivalate (43);
3-(((((1R,2S,5R)-2-carbamoyl-7-oxo-1,6-diazabicyclo[3.2.1]octan-6-yl)oxy)sulfonyl)oxy)-2,2-dimethylpropyl 3-chloro-2,6-dimethoxybenzoate (44);
4-(((((1R,2S,5R)-2-carbamoyl-7-oxo-1,6-diazabicyclo[3.2.1]octan-6-yl)oxy)sulfonyl)oxy)-2,2,3,3-tetramethylbutyl 2,6-dimethylbenzoate (45);
4-(((((1R,2S,5R)-2-carbamoyl-7-oxo-1,6-diazabicyclo[3.2.1]octan-6-yl)oxy)sulfonyl)oxy)-2,2,3,3-tetramethylbutyl benzoate (46);
4-(((((1R,2S,5R)-2-carbamoyl-7-oxo-1,6-diazabicyclo[3.2.1]octan-6-yl)oxy)sulfonyl)oxy)-2,2,3,3-tetramethylbutyl propionate (47);
(1R,2S,5R)-2-carbamoyl-7-oxo-1,6-diazabicyclo[3.2.1]octan-6-yl ((3-methyl-2-oxotetrahydro-2H-pyran-3-yl)methyl) sulfate (48);
2-(3-(((((1R,2S,5R)-2-carbamoyl-7-oxo-1,6-diazabicyclo[3.2.1]octan-6-yl)oxy)sulfonyl)oxy)-2,2-dimethylpropyl)phenyl acetate (49);
2-(3-(((((1R,2S,5R)-2-carbamoyl-7-oxo-1,6-diazabicyclo[3.2.1]octan-6-yl)oxy)sulfonyl)oxy)-2,2-dimethylpropyl)phenyl pivalate (50);
S-(4-(((((1R,2S,5R)-2-carbamoyl-7-oxo-1,6-diazabicyclo[3.2.1]octan-6-yl)oxy)sulfonyl)oxy)-3,3-dimethylbutyl) ethanethioate (51);
S-(5-(((((1R,2S,5R)-2-carbamoyl-7-oxo-1,6-diazabicyclo[3.2.1]octan-6-yl)oxy)sulfonyl)oxy)-4,4-dimethylpentyl) ethanethioate (52);
S-(3-(((((1R,2S,5R)-2-carbamoyl-7-oxo-1,6-diazabicyclo[3.2.1]octan-6-yl)oxy)sulfonyl)oxy)-2,2-dimethylpropyl) ethanethioate (53);
3-(((((1R,2S,5R)-2-carbamoyl-7-oxo-1,6-diazabicyclo[3.2.1]octan-6-yl)oxy)sulfonyl)oxy)-2,2-dimethylpropyl 2,6-dimethylbenzoate (54);
3-(((((1R,2S,5R)-2-carbamoyl-7-oxo-1,6-diazabicyclo[3.2.1]octan-6-yl)oxy)sulfonyl)oxy)-2,2-dimethylpropyl adamantane-1-carboxylate (55);
diethyl 2-((((((1R,2S,5R)-2-carbamoyl-7-oxo-1,6-diazabicyclo[3.2.1]octan-6-yl)oxy)sulfonyl)oxy)methyl)-2-methylmalonate (56);
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate (57);
butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (58);
(5-methyl-2-oxo-1,3-dioxol-4-yl)methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (59);
4-(((((1R,2S,5R)-2-carbamoyl-7-oxo-1,6-diazabicyclo[3.2.1]octan-6-yl)oxy)sulfonyl)oxy)-3,3-dimethylbutyl pivalate (60);
ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate (61);
4-(((((1R,2S,5R)-2-carbamoyl-7-oxo-1,6-diazabicyclo[3.2.1]octan-6-yl)oxy)sulfonyl)oxy)-3,3-dimethylbutyl 2,6-dimethylbenzoate (62);
4-(((((1R,2S,5R)-2-carbamoyl-7-oxo-1,6-diazabicyclo[3.2.1]octan-6-yl)oxy)sulfonyl)oxy)-3,3-dimethylbutyl adamantane-1-carboxylate (63);
4-(((((1R,2S,5R)-2-carbamoyl-7-oxo-1,6-diazabicyclo[3.2.1]octan-6-yl)oxy)sulfonyl)oxy)-3,3-dimethylbutyl 2,6-dimethoxybenzoate (64);
5-(((((1R,2S,5R)-2-carbamoyl-7-oxo-1,6-diazabicyclo[3.2.1]octan-6-yl)oxy)sulfonyl)oxy)-4,4-dimethylpentyl benzoate (65);
5-(((((1R,2S,5R)-2-carbamoyl-7-oxo-1,6-diazabicyclo[3.2.1]octan-6-yl)oxy)sulfonyl)oxy)-4,4-dimethylpentyl 2,6-dimethoxybenzoate (66);
5-(((((1R,2S,5R)-2-carbamoyl-7-oxo-1,6-diazabicyclo[3.2.1]octan-6-yl)oxy)sulfonyl)oxy)-4,4-dimethylpentyl 2,6-dimethylbenzoate (67);
5-(((((1R,2S,5R)-2-carbamoyl-7-oxo-1,6-diazabicyclo[3.2.1]octan-6-yl)oxy)sulfonyl)oxy)-4,4-dimethylpentyl 2-methylbenzoate (68);
4-(((((1R,2S,5R)-2-carbamoyl-7-oxo-1,6-diazabicyclo[3.2.1]octan-6-yl)oxy)sulfonyl)oxy)-2,2,3,3-tetramethylbutyl 3-chloro-2,6-dimethoxybenzoate (69);
2-((((((1R,2S,5R)-2-carbamoyl-7-oxo-1,6-diazabicyclo[3.2.1]octan-6-yl)oxy)sulfonyl)oxy)methyl)-2-methylpropane-1,3-diyl dibenzoate (70);
2-((((((1R,2S,5R)-2-carbamoyl-7-oxo-1,6-diazabicyclo[3.2.1]octan-6-yl)oxy)sulfonyl)oxy)methyl)-2-methylpropane-1,3-diyl diacetate (71);
5-(((((1R,2S,5R)-2-carbamoyl-7-oxo-1,6-diazabicyclo[3.2.1]octan-6-yl)oxy)sulfonyl)oxy)-2,2,4,4-tetramethylpentyl 2,6-dimethoxybenzoate (72);
ethyl 3-(((((1R,2S,5R)-2-carbamoyl-7-oxo-1,6-diazabicyclo[3.2.1]octan-6-yl)oxy)sulfonyl)oxy)-2,2-dimethylbutanoate (73);
(1R,2S,5R)-2-carbamoyl-7-oxo-1,6-diazabicyclo[3.2.1]octan-6-yl ((3,5,5-trimethyl-2-oxotetrahydrofuran-3-yl)methyl) sulfate (74);
   a pharmaceutically acceptable salt of any of the foregoing; and
   a combination of any of the foregoing.

In compounds of Formula (1), the compound can be selected from:
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3);
benzyl 3-(((((1R,2S,5R)-2-carbamoyl-7-oxo-1,6-diazabicyclo[3.2.1]octan-6-yl)oxy)sulfonyl)oxy)-2,2-dimethylpropanoate (4);
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (10);
isopropyl 3-(((((1R,2S,5R)-2-carbamoyl-7-oxo-1,6-diazabicyclo[3.2.1]octan-6-yl)oxy)sulfonyl)oxy)-2,2-dimethylpropanoate (11);
hexyl 3-(((((1R,2S,5R)-2-carbamoyl-7-oxo-1,6-diazabicyclo[3.2.1]octan-6-yl)oxy)sulfonyl)oxy)-2,2-dimethylpropanoate (12);
heptyl 3-(((((1R,2S,5R)-2-carbamoyl-7-oxo-1,6-diazabicyclo[3.2.1]octan-6-yl)oxy)sulfonyl)oxy)-2,2-dimethylpropanoate (13);
tert-butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (14);
2-methoxyethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (15);
oxetan-3-yl 3-(((((1R,2S,5R)-2-carbamoyl-7-oxo-1,6-diazabicyclo[3.2.1]octan-6-yl)oxy)sulfonyl)oxy)-2,2-dimethylpropanoate (16);
ethyl 1-((((((1R,2S,5R)-2-carbamoyl-7-oxo-1,6-diazabicyclo[3.2.1]octan-6-yl)oxy)sulfonyl)oxy)methyl)cyclohexanecarboxylate (17);
ethyl 1-((((((1R,2S,5R)-2-carbamoyl-7-oxo-1,6-diazabicyclo[3.2.1]octan-6-yl)oxy)sulfonyl)oxy)methyl)cyclopropanecarboxylate (18);
ethyl 1-((((((1R,2S,5R)-2-carbamoyl-7-oxo-1,6-diazabicyclo[3.2.1]octan-6-yl)oxy)sulfonyl)oxy)methyl)cyclobutanecarboxylate (19);
hexyl 5-(((((1R,2S,5R)-2-carbamoyl-7-oxo-1,6-diazabicyclo[3.2.1]octan-6-yl)oxy)sulfonyl)oxy)-4,4-dimethylpentanoate (36);
heptyl 5-(((((1R,2S,5R)-2-carbamoyl-7-oxo-1,6-diazabicyclo[3.2.1]octan-6-yl)oxy)sulfonyl)oxy)-4,4-dimethylpentanoate (37);
(1R,2S,5R)-2-carbamoyl-7-oxo-1,6-diazabicyclo[3.2.1]octan-6-yl ((3-methyl-2-oxotetrahydrofuran-3-yl)methyl) sulfate (42);
S-(3-(((((1R,2S,5R)-2-carbamoyl-7-oxo-1,6-diazabicyclo[3.2.1]octan-6-yl)oxy)sulfonyl)oxy)-2,2-dimethylpropyl) ethanethioate (53);
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate (57);
butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (58);
(5-methyl-2-oxo-1,3-dioxol-4-yl)methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (59);
   a pharmaceutically acceptable salt of any of the foregoing; and
   a combination of any of the foregoing.

In compounds of Formula (1), the compound can be selected from:
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3);
benzyl 3-(((((1R,2S,5R)-2-carbamoyl-7-oxo-1,6-diazabicyclo[3.2.1]octan-6-yl)oxy)sulfonyl)oxy)-2,2-dimethylpropanoate (4);
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (10);
isopropyl 3-(((((1R,2S,5R)-2-carbamoyl-7-oxo-1,6-diazabicyclo[3.2.1]octan-6-yl)oxy)sulfonyl)oxy)-2,2-dimethylpropanoate (11);
hexyl 3-(((((1R,2S,5R)-2-carbamoyl-7-oxo-1,6-diazabicyclo[3.2.1]octan-6-yl)oxy)sulfonyl)oxy)-2,2-dimethylpropanoate (12);
heptyl 3-(((((1R,2S,5R)-2-carbamoyl-7-oxo-1,6-diazabicyclo[3.2.1]octan-6-yl)oxy)sulfonyl)oxy)-2,2-dimethylpropanoate (13);
tert-butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (14);
2-methoxyethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (15);
oxetan-3-yl 3-(((((1R,2S,5R)-2-carbamoyl-7-oxo-1,6-diazabicyclo[3.2.1]octan-6-yl)oxy)sulfonyl)oxy)-2,2-dimethylpropanoate (16);
ethyl 1-((((((1R,2S,5R)-2-carbamoyl-7-oxo-1,6-diazabicyclo[3.2.1]octan-6-yl)oxy)sulfonyl)oxy)methyl)cyclohexanecarboxylate (17);
ethyl 1-((((((1R,2S,5R)-2-carbamoyl-7-oxo-1,6-diazabicyclo[3.2.1]octan-6-yl)oxy)sulfonyl)oxy)methyl)cyclopropanecarboxylate (18);
ethyl 1-((((((1R,2S,5R)-2-carbamoyl-7-oxo-1,6-diazabicyclo[3.2.1]octan-6-yl)oxy)sulfonyl)oxy)methyl)cyclobutanecarboxylate (19);
   a pharmaceutically acceptable salt of any of the foregoing; and
   a combination of any of the foregoing.

In compounds of Formula (1), the compound can be selected from:
hexyl 5-(((((1R,2S,5R)-2-carbamoyl-7-oxo-1,6-diazabicyclo[3.2.1]octan-6-yl)oxy)sulfonyl)oxy)-4,4-dimethylpentanoate (36);
heptyl 5-(((((1R,2S,5R)-2-carbamoyl-7-oxo-1,6-diazabicyclo[3.2.1]octan-6-yl)oxy)sulfonyl)oxy)-4,4-dimethylpentanoate (37);
(1R,2S,5R)-2-carbamoyl-7-oxo-1,6-diazabicyclo[3.2.1]octan-6-yl ((3-methyl-2-oxotetrahydrofuran-3-yl)methyl) sulfate (42);
S-(3-(((((1R,2S,5R)-2-carbamoyl-7-oxo-1,6-diazabicyclo[3.2.1]octan-6-yl)oxy)sulfonyl)oxy)-2,2-dimethylpropyl) ethanethioate (53);
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate (57);
butyl 3-(((((1R,2S,5R)-2-carbamoyl-7-oxo-1,6-diazabicyclo[3.2.1]octan-6-yl)oxy)sulfonyl)oxy)-2,2-dimethylpropanoate (58);
(5-methyl-2-oxo-1,3-dioxol-4-yl)methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (59);
   a pharmaceutically acceptable salt of any of the foregoing; and
   a combination of any of the foregoing.

In a compound of Formula (1),
each R¹ can be independently selected from C₁₋₃ alkyl, or each R¹ together with the geminal carbon atom to which they are bonded form a C₃₋₆ cycloalkyl ring, a substituted C₃₋₆ cycloalkyl ring, a C₃₋₆ heterocycloalkyl ring, or a substituted C₃₋₆ heterocycloalkyl ring;
R² can be a single bond;
R³ can be —C(O)—O—R⁴; and
R⁴ can be selected from C₁₋₈ alkyl, C₁₋₈ heteroalkyl, C₇₋₉ arylalkyl, C₅₋₇ heterocycloalkyl, substituted C₁₋₈ alkyl, substituted C₁₋₈ heteroalkyl, substituted C₇₋₉ arylalkyl, and substituted C₅₋₇ heterocycloalkyl.

In a compound of Formula (1),
each R¹ can be independently selected from C₁₋₃ alkyl, or each R¹ together with the carbon atom to which they are bonded form a C₃₋₆ cycloalkyl ring;
R² can be selected from single bond, methane-diyl, and ethane-diyl; and
R³ can be selected from —C(O)—O—R⁴ and —S—C(O)—R⁴, wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In a compound of Formula (1),
each R¹ can be independently selected from C₁₋₃ alkyl, or each R¹ together with the carbon atom to which they are bonded form a C₃₋₆ cycloalkyl ring;
R² can be a single bond; and
R³ can be —C(O)—O—R⁴, where R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In a compound of Formula (1),
each R¹ can be independently selected from C₁₋₃ alkyl, or each R¹ together with the carbon atom to which they are bonded form a C₃₋₆ cycloalkyl ring;
R² can be —(CH₂)₂—; and
R³ can be —C(O)—O—R⁴ wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In a compound of Formula (1),
each R¹ can be selected from C₁₋₃ alkyl, or each R¹ together with the carbon atom to which they are bonded form a C₃₋₆ cycloalkyl ring;
R² can be —CH₂—; and
R³ can be —S—C(O)—R⁴, wherein R⁴ can be selected from C₁₋₁₀ alkyl, C₁₋₁₀ heteroalkyl, CO₅₋₁₀ arylalkyl, C₃₋₆ heterocycloalkyl, substituted C₄₋₁₀ heterocycloalkylalkyl.

In a compound of Formula (1),
each R¹ together with the carbon atom to which they are bonded form a C₃₋₆ cycloalkyl ring, a C₃₋₆ heterocycloalkyl ring, a C₃₋₆ cycloalkyl ring, or a C₃₋₆ heterocycloalkyl ring;
R² can be a single bond; and
R³ can be C₁₋₃ alkyl.

In a compound of Formula (1),
each R¹ can be independently selected from C₁₋₃ alkyl;
R² can be selected from a single bond and methanediyl; and
R³ can be selected from —O—C(O)—R⁴ and —C(O)—O—R⁴, wherein R⁴ can be selected from C₁₋₁₀ alkyl and substituted phenyl.

In a compound of Formula (1),
each R¹ can be independently selected from C₁₋₃ alkyl;
R² can be a single bond;
R³ can be —CH═C(R⁴)₂, wherein each R⁴ can be —C(O)—O—R⁸, or each R⁴ together with the carbon atom to which they are bonded from a substituted heterocyclohexyl ring; and
each R⁸ can be C₁₋₄ alkyl.

In a compound of Formula (1),
each R¹ can be independently selected from C₁₋₃ alkyl;
R² can be selected from a single bond and methanediyl; and
R³ can be substituted phenyl, wherein the one or more substituents can be independently selected from —CH₂—O—C(O)—R⁴ and —O—C(O)—R⁴, wherein R⁴ can be selected from C₁₋₁₀ alkyl and phenyl.

In a compound of Formula (1),
each R¹ can be independently selected from C₁₋₃ alkyl;
R² can be selected from —C(R⁸)₂— and —CH₂—C(R⁸)₂—, wherein each R⁸ can be independently selected from C₁₋₃ alkyl; and
R³ can be selected from —C(O)—O—R⁴ and —O—C(O)—R⁴, wherein R⁴ can be selected from C₁₋₁₀ alkyl, C₁₋₁₀ heteroalkyl, substituted C₁₋₁₀ alkyl, substituted C₁₋₁₀ heteroalkyl, and 4(yl-methyl)-5-methyl-1,3-dioxol-2-one.

In a compound of Formula (1),
each R¹ together with the carbon atom to which they are bonded form a substituted C₅₋₆ heterocyclic ring;
R² can be a single bond; and
R³ can be C₁₋₃ alkyl.

A compound of Formula (1) can be a compound of sub-genus (1A), or a pharmaceutically acceptable salt thereof, wherein,
each R¹ can be independently selected from C₁₋₃ alkyl, or each R¹ together with the carbon atom to which they are bonded form a C₃₋₆ cycloalkyl ring;
R² can be selected from single bond, methane-diyl, and ethane-diyl; and
R³ can be selected from —C(O)—O—R⁴ and —S—C(O)—R⁴, wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In compounds of subgenus (1A), each R¹ can be independently selected from C₁₋₃ alkyl.

In compounds of subgenus (1A), each R¹ together with the carbon atom to which they are bonded form a C₃₋₆ cycloalkyl ring.

In compounds of subgenus (1A), R² a single bond.

In compounds of subgenus (1A), R² can be methane-diyl.

In compounds of subgenus (1A), R² can be ethane-diyl.

In compounds of subgenus (1A), R³ can be —C(O)—O—R⁴.

In compounds of subgenus (1A), R³ can be —S—C(O)—R⁴.

In compounds of subgenus (1A), R⁴ can be C₁₋₁₀ alkyl.

In compounds of subgenus (1A), R⁴ can be C₁₋₁₀ heteroalkyl.

In compounds of subgenus (1A), R⁴ can be C₅₋₁₀ arylalkyl.

In compounds of subgenus (1A), R⁴ can be C₃₋₆ heterocycloalkyl.

In compounds of subgenus (1A), R⁴ can be substituted C₄₋₁₀ heterocycloalkylalkyl.

A compound of Formula (1) can be a compound of sub-genus (1B), or a pharmaceutically acceptable salt thereof, wherein,
each R¹ can be independently selected from C₁₋₃ alkyl, or each R¹ together with the carbon atom to which they are bonded form a C₃₋₆ cycloalkyl ring;
R² can be a single bond; and
R³ can be —C(O)—O—R⁴, where R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In compounds of subgenus (1B), each R¹ can be independently selected from C₁₋₃ alkyl.

In compounds of subgenus (1B), each R¹ together with the carbon atom to which they are bonded form a C₃₋₆ cycloalkyl ring.

In compounds of subgenus (1B), R⁴ can be selected from C₁₋₇ alkyl, C₁₋₁₀ heteroalkyl wherein the one or more heteroatoms can be oxygen, —CH₂—C₄₋₆ cycloalkyl, —(CH₂)₂—C₄₋₆ cycloalkyl, C₃₋₆ heterocycloalkyl wherein the one or more heteroatoms can be oxygen, and —CH₂—C₃₋₆ substituted heterocycloalkyl, and —(CH₂)₂—C₃₋₆ substituted heterocycloalkyl.

In compounds of subgenus (1B), in the substituted C₃₋₆ heterocycloalkyl the one or more heteroatoms can be oxygen, and the one or more substituents can be independently selected from C₁₋₃ alkyl and ═O.

In compounds of subgenus (1B), each R¹ can be methyl, or each R¹ together with the carbon atom to which they are bonded form a cyclohexyl ring or a cyclopentyl ring.

In compounds of subgenus (1B), R⁴ can be selected from methyl, ethyl, n-propyl, iso propyl, n-butyl, n-hexyl, n-heptyl, —CH₂—CH₂—O—CH₃, benzyl, 3-oxetanyl, and methyl-5-methyl-1,3-dioxol-2-one.

In compounds of subgenus (1B),
each R¹ can be methyl, or each R¹ together with the carbon atom to which they are bonded form a cyclohexyl ring or a cyclopentyl ring;
R² can be a single bond; and
R³ can be —C(O)—O—R⁴, wherein R⁴ can be selected from methyl, ethyl, n-propyl, iso propyl, n-butyl, n-hexyl, n-heptyl, —CH₂—CH₂—O—CH₃, —CH₂-phenyl (benzyl), 3-oxetanyl, and methyl-5-methyl-1,3-dioxol-2-one.

A compound of Formula (1) can be a compound of sub-genus (1C), or a pharmaceutically acceptable salt thereof, wherein,
each R¹ can be independently selected from C₁₋₃ alkyl, or each R¹ together with the carbon atom to which they are bonded form a C₃₋₆ cycloalkyl ring;
R² can be —(CH₂)₂—; and
R³ can be —C(O)—O—R⁴ wherein R⁴ can be selected from C₁₋₁₀ alkyl, C₁₋₁₀ heteroalkyl, C₅₋₁₀ arylalkyl, C₃₋₆ heterocycloalkyl, and substituted C₄₋₁₀ heterocycloalkylalkyl.

In compounds of subgenus (1C), each R¹ can be independently selected from C₁₋₃ alkyl.

In compounds of subgenus (1C), each R¹ together with the carbon atom to which they are bonded form a C₃₋₆ cycloalkyl ring.

In compounds of subgenus (1C), R⁴ can be selected from C₁₋₇ alkyl, C₁₋₁₀ heteroalkyl wherein the one or more heteroatoms can be oxygen, —CH₂—C₄₋₆ cycloalkyl, —(CH₂)₂—C₄₋₆ cycloalkyl, C₃₋₆ heterocycloalkyl wherein the one or more heteroatoms can be oxygen, —CH₂—C₃₋₆ substituted heterocycloalkyl, and —(CH₂)₂—C₃₋₆ substituted heterocycloalkyl.

In compounds of subgenus (1C), in the substituted C₃₋₆ heterocycloalkyl the one or more heteroatoms can be oxygen, and the one or more substituents can be independently selected from C₁₋₃ alkyl and ═O.

In compounds of subgenus (1C), R⁴ can be C₁₋₁₀ alkyl.

In compounds of subgenus (1C),
each R¹ can be methyl;
R² can be —(CH₂)₂—; and
R³ can be —C(O)—O—R⁴ wherein R⁴ can be selected from n-hexyl and n-heptyl.

A compound of Formula (1) can be a compound of sub-genus (1D), or a pharmaceutically acceptable salt thereof, wherein,
each R¹ can be selected from C₁₋₃ alkyl, or each R¹ together with the carbon atom to which they are bonded form a C₃₋₆ cycloalkyl ring;
R² can be —CH₂—; and
R³ can be —S—C(O)—R⁴, wherein R⁴ can be selected from C₁₋₁₀ alkyl, C₁₋₁₀ heteroalkyl, CO₅₋₁₀ arylalkyl, C₃₋₆ heterocycloalkyl, and substituted C₄₋₁₀ heterocycloalkylalkyl.

In compounds of subgenus (1D), each R¹ can be independently selected from C₁₋₃ alkyl.

In compounds of subgenus (1D), each R¹ together with the carbon atom to which they are bonded form a C₃₋₆ cycloalkyl ring.

In compounds of subgenus (1D), R⁴ can be selected from C₁₋₇ alkyl, C₁₋₁₀ heteroalkyl wherein the one or more heteroatoms can be oxygen, —CH₂—C₄₋₆ cycloalkyl, —(CH₂)₂—C₄₋₆ cycloalkyl, C₃₋₆ heterocycloalkyl wherein the one or more heteroatoms can be oxygen, —CH₂—C₃₋₆ substituted heterocycloalkyl, and —(CH₂)₂—C₃₋₆ substituted heterocycloalkyl.

In compounds of subgenus (1D), in the substituted C₃₋₆ heterocycloalkyl the one or more heteroatoms can be oxygen, and the one or more substituents can be independently selected from C₁₋₃ alkyl and ═O.

In compounds of subgenus (1D), R⁴ can be C₁₋₁₀ alkyl.

In compounds of subgenus (1D),
each R¹ can be methyl;
R² can be —CH₂—; and
R³ can be —S—C(O)—R⁴, wherein R⁴ can be methyl.

A compound of Formula (1) can be a compound of sub-genus (1E), or a pharmaceutically acceptable salt thereof, wherein,
each R¹ together with the carbon atom to which they are bonded form a C₃₋₆ cycloalkyl ring, a C₃₋₆ heterocycloalkyl ring, a C₃₋₆ cycloalkyl ring, or a C₃₋₆ heterocycloalkyl ring;
R² can be a single bond; and
R³ can be C₁₋₃ alkyl.

In compounds of subgenus (1E), each R¹ together with the carbon atom to which they are bonded form a C₃₋₆ heterocycloalkyl ring or a C₃₋₆ heterocycloalkyl ring.

In compounds of subgenus (1E), the one or more heteroatoms can be oxygen and the one or more substituents can be ═O.

In compounds of subgenus (1E),
each R¹ together with the carbon atom to which they are bonded form a dihydrofuran-2(3H)-one ring;
R² can be a single bond; and
R³ can be methyl.

A compound of Formula (1) can be a compound of sub-genus (1F), or a pharmaceutically acceptable salt thereof, wherein,
each R¹ can be independently selected from C₁₋₃ alkyl;
R² can be selected from a single bond and methanediyl; and
R³ can be selected from —O—C(O)—R⁴ and —C(O)—O—R⁴, wherein R⁴ can be selected from C₁₋₁₀ alkyl and substituted phenyl.

In compounds of subgenus (1F), R² can be a single bond.

In compounds of subgenus (1F), R² can be methanediyl.

In compounds of subgenus (1F), R³ can be —O—C(O)—R⁴.

In compounds of subgenus (1F), R² can be methanediyl; and R³ can be —O—C(O)—R⁴.

In compounds of subgenus (1F), R³ can be —C(O)—O—R⁴.

In compounds of subgenus (1F), R² can be a single bond; and R³ can be —C(O)—O—R⁴.

In compounds of subgenus (1E), R² can be a single bond; R³ can be —C(O)—O—R⁴; and R⁴ can be C₁₋₃ alkyl.

In compounds of subgenus (1F), R⁴ can be C₁₋₁₀ alkyl.

In compounds of subgenus (1F), R⁴ can be C₁₋₄ alkyl.

In compounds of subgenus (1F), R⁴ can be substituted phenyl.

In compounds of subgenus (1F), R² can be methanediyl; R³ can be —O—C(O)—R⁴; and R⁴ can be substituted phenyl.

In compounds of subgenus (1F), the one or more substituents can be independently selected from halogen, C₁₋₃ alkyl, and C₁₋₃ alkoxy.

In compounds of subgenus (1F), the substituted phenyl can be 2,6-substituted phenyl.

In compounds of subgenus (1F), each of the substituents can be selected from C₁₋₃ alkyl and C₁₋₃ alkoxy.

In compounds of subgenus (1F), the substituted phenyl can be 2,5,6-substituted phenyl.

In compounds of subgenus (1F), each of the substituents at the 2 and 6 positions can be independently selected from C₁₋₃ alkyl and C₁₋₃ alkoxy; and the substituent at the 5 position can be halogen.

A compound of Formula (1) can be a compound of sub-genus (1G), or a pharmaceutically acceptable salt thereof, wherein,
each R¹ can be independently selected from C₁₋₃ alkyl;
R² can be a single bond;
R³ can be —CH═C(R⁴)₂, wherein each R⁴ can be —C(O)—O—R⁸, or each R⁴ together with
the carbon atom to which they are bonded from a substituted heterocyclohexyl ring; and each R⁸ can be C₁₋₄ alkyl.

In compounds of subgenus (1G), each R⁴ can be —C(O)—O—R⁸.

In compounds of subgenus (1G), each R⁴ can be —C(O)—O—R⁸, or each R⁴ together with the carbon atom to which they are bonded from a substituted heterocyclohexyl ring.

In compounds of subgenus (1G), in the substituted heterocyclohexyl ring, the one or more heteroatoms can be oxygen.

In compounds of subgenus (1G), in the substituted heterocyclohexyl ring, the one or more substituents can be independently selected from C₁₋₃ alkyl and ═O.

In compounds of subgenus (1G), the substituted heterocycloalkyl ring can be 2,2-dimethyl-5-yl-1,3-dioxane-4,6-dione.

A compound of Formula (1) can be a compound of sub-genus (1H), or a pharmaceutically acceptable salt thereof, wherein,
each R¹ can be independently selected from C₁₋₃ alkyl;
R² can be selected from a single bond and methanediyl; and
R³ can be substituted phenyl, wherein the one or more substituents can be independently selected from —CH₂—O—C(O)—R⁴ and —O—C(O)—R⁴, wherein R⁴ can be selected from C₁₋₁₀ alkyl and phenyl.

In compounds of subgenus (1H), R² can be a single bond.

In compounds of subgenus (1H), R² can be 2-substituted phenyl.

In compounds of subgenus (1H), the one or more substituents can be —CH₂—O—C(O)—R⁴.

In compounds of subgenus (1H), the one or more substituents can be —O—C(O)—R⁴.

In compounds of subgenus (1H), R⁴ can be C₁₋₁₀ alkyl.

In compounds of subgenus (1H), R⁴ can be selected from methyl, ethyl, iso-propyl, pivaloyl, and phenyl.

A compound of Formula (1) can be a compound of sub-genus (11), or a pharmaceutically acceptable salt thereof, wherein,
each R¹ can be independently selected from C₁₋₃ alkyl;
R² can be selected from —C(R⁸)₂— and —CH₂—C(R⁸)₂—, wherein each R⁸ can be independently selected from C₁₋₃ alkyl; and
R³ can be selected from —C(O)—O—R⁴ and —O—C(O)—R⁴, wherein R⁴ can be selected from C₁₋₁₀ alkyl, C₁₋₁₀ heteroalkyl, substituted C₁₋₁₀ alkyl, substituted C₁₋₁₀ heteroalkyl, and 4(yl-methyl)-5-methyl-1,3-dioxol-2-one.

In compounds of subgenus (11), each R¹ can be methyl.

In compounds of subgenus (11), R² can be —C(R⁸)₂—.

In compounds of subgenus (11), R² can be —CH₂—C(R⁸)₂—.

In compounds of subgenus (11), each R¹ can be methyl.

In compounds of subgenus (11), each R¹ can be methyl; and each R⁸ can be methyl.

In compounds of subgenus (11), R³ can be —C(O)—O—R⁴.

In compounds of subgenus (11), R³ can be —O—C(O)—R⁴.

A compound of Formula (1) can be a compound of sub-genus (1J), or a pharmaceutically acceptable salt thereof, wherein,
each R¹ together with the carbon atom to which they are bonded form a substituted C₅₋₆ heterocyclic ring;
R² can be a single bond; and
R³ can be C₁₋₃ alkyl.

In compounds of subgenus (1J), in the substituted C₅₋₆ heterocyclic ring, the one or more heteroatoms can be oxygen; and the one or more substituents can be independently selected from C₁₋₃ alkyl and ═O.

In compounds of subgenus (1J), each R¹ together with the carbon atom to which they are bonded form a tetrahydro-2H-pyran-2-one ring.

In compounds of subgenus (1J),
each R¹ can be independently selected from C₁₋₃ alkyl;
R² can be selected from C₂₋₄ alkanediyl; and
R³ can be substituted C₅₋₆ heterocycloalkyl, wherein the one or more heteroatoms can be independently selected from N and O; and the one or more substituents can be independently selected from C₁₋₃ alkyl and ═O.

In compounds of subgenus (1J), R³ can have the structure of Formula (3):

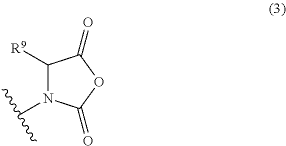

wherein R⁹ can be selected from hydrogen, C₁₋₆ alkyl, C₄₋₆ cycloalkyl, C₁₋₆ heteroalkyl, C₄₋₆ heterocycloalkyl, substituted C₁₋₆ alkyl, substituted C₄₋₆ cycloalkyl, substituted C₁₋₆ heteroalkyl, and substituted C₄₋₆ heterocycloalkyl.

In compounds of subgenus (1J), R⁹ can be selected from hydrogen and C₁₋₆ alkyl such as C₁₋₄ alkyl such as methyl or ethyl.

An avibactam derivative provided by the present disclosure can include compounds of

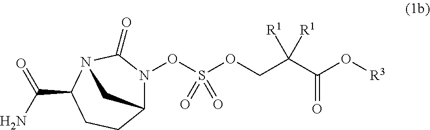

or a pharmaceutically acceptable salt thereof, wherein, each R¹ can independently be selected from C₁₋₆ alkyl; and R³ can be C₁₋₆ alkyl.

In avibactam derivatives of Formula (1b), each R¹ can independently be C₁₋₃ alkyl, and R³ can be C₁₋₃ alkyl.

In avibactam derivatives of Formula (1b), each R¹ can be methyl, and R³ can be C₁₋₃ alkyl such as methyl, ethyl, n-propyl, or iso-propyl.

An avibactam derivative can be selected from:
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
methyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
propyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
propyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
   a pharmaceutically acceptable salt of any of the foregoing; and
   a combination of any of the foregoing.

An avibactam derivative can be avibactam derivative (10), methyl 3-(((((1R,2S,5R)-2-carbamoyl-7-oxo-1,6-diazabicyclo[3.2.1]octan-6-yl)oxy)sulfonyl)oxy)-2,2-dimethylpropanoate (10), having the structure:

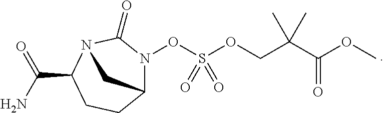

An avibactam derivative can be avibactam derivative (3), ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), has the structure:

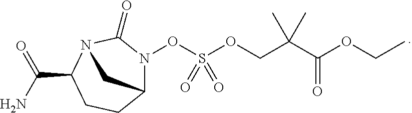

A compound of Formula (1), (1a), or (1b) can be a solvate, a pharmaceutically acceptable salt, or a combination thereof.

In compounds of Formula (1), (1a), or (1b), a pharmaceutically acceptable salt can be the hydrochloride salt.

In compounds of Formula (1), (1a), or (1b), a pharmaceutically acceptable salt can be the dihydrochloride salt.

A compound of Formula (1), (1a), or (1b) can be a pharmaceutically acceptable salt of a compound of Formula (1), a hydrate thereof, or a solvate of any of the foregoing.

The avibactam derivatives described herein can be synthesized using the methods described in U.S. Pat. No. 10,085,999.

Pharmaceutical compositions provided by the present disclosure can be administered orally.

Avibactam derivatives, when orally administered, provide an enhanced oral bioavailability of the β-lactamase inhibitor compared to the oral bioavailability of the parent β-lactamase inhibitor. For example, avibactam derivatives of Formula (1) can exhibit an avibactam oral bioavailability (% F) of at least 10%, at least 20%, at least 30%, at least 40%, at least 50%, at least 60%, at least 70%, or at least 80%. The oral bioavailability of avibactam in a human is about 7%.

As disclosed in U.S. Pat. No. 10,085,999, avibactam derivatives (3), (4), (10), (11), (12), (13), (14), (15), (16), (17), (18), and (19) exhibit an oral bioavailability (% F) greater than 10%. Also, compounds (36), (37), (42), (53), (57), (58), and (59) exhibit an avibactam oral bioavailability (% F) in Sprague-Dawley rats greater than 10%. In similar studies avibactam exhibits an oral bioavailability (% F) in Sprague-Dawley rats of 1.2%. Avibactam derivatives (3), (13), and (15) exhibited an avibactam oral bioavailability in male Beagle dogs and in Cynomolgus monkeys of greater than 50% F. Avibactam derivative (3) exhibits an avibactam oral bioavailability in human patients greater than 50% F.

Pharmaceutical compositions provided by the present disclosure can comprise amoxicillin or a pharmaceutically acceptable salt thereof and an avibactam derivative of Formula (1) or a pharmaceutically acceptable salt thereof.

Pharmaceutical compositions provided by the present disclosure can comprise a therapeutically effective amount of amoxicillin or a pharmaceutically acceptable salt thereof and a therapeutically effective amount of an avibactam derivative of Formula (1), i.e. an avibactam derivative provided by the present disclosure, or a pharmaceutically acceptable salt thereof.

A pharmaceutical composition can comprise a pharmaceutically acceptable carrier or excipient, or a combination of pharmaceutically acceptable carriers or excipients.

A pharmaceutical composition can comprise an oral formulation. An oral formulation can be, for example, in the form of liquid or solid dosage form. A solid dosage form for oral administration can be in the form of solutions, suspensions, capsules, tablets, powders, pills, or granules. An oral solid dosage form can comprise, for example, fillers, extenders, binders, humectants, disintegrating agents, absorption accelerators, wetting agents, absorbents, lubricants, buffering agents, or combinations of any of the foregoing. Examples of liquid oral dosage forms include soft gel capsules containing a liquid, oral suspensions, syrups, and elixirs.

An oral dosage form can comprise a therapeutically effective amount of amoxicillin or a pharmaceutically acceptable salt thereof and an avibactam derivative of Formula (1) or a pharmaceutically acceptable salt thereof. An oral dosage form can comprise a fraction of therapeutically effective amount of amoxicillin or a pharmaceutically acceptable salt thereof and/or a fraction of a therapeutically effective amount avibactam derivative of Formula (1) or a pharmaceutically acceptable salt thereof. Oral dosage forms containing a fractional therapeutically effective amount of amoxicillin and/or an avibactam derivative can be intended to be administered simultaneously as multiple dosage forms that in total provide a therapeutically effective amount or can be intended to be administered over a period of time such as, for example, from 1 to 6 times daily to provide a therapeutically effective amount of amoxicillin and avibactam in the plasma of a patient.

Amoxicillin and an avibactam derivative can be provided in separate dosage forms or can be combined in a single dosage form.

Amoxicillin and an avibactam derivative can be co-formulated such that the compounds are homogeneously distributed throughout an oral dosage form.

Amoxicillin and an avibactam derivative can be sequestered in different portions of an oral dosage form. For example, one or both compounds can be contained within particulates dispersed in a carrier, or the compounds can be independently dispersed within separate portions of the oral dosage form such as, for example, to form a core-shell structure.

An oral dosage form comprising both amoxicillin and an avibactam derivative can comprise a weight ratio of amoxicillin to avibactam equivalents within a range, for example, from 1:1 to 1:4, from 1:1 to 1:3, from 1:1 to 1:2, from 1:1 to 1:1.5, from 1:1 to 1:0.9, or from 1:1 to 1:0.5.

An oral dosage form can comprise, for example, from 100 mg to 1,400 mg of amoxicillin, from 100 mg to 1,200 mg, from 100 mg to 1,000 mg, from 100 mg to 800 mg, or from 100 mg to 600 mg of amoxicillin.

Current FDA oral dosages of amoxicillin are 500 mg to 1,000 mg TID, 1,000 mg BID, and 1,000 mg q6 h is also used. An oral dosage form such as a tablet or capsule can comprise, for example, from 100 mg to 1,000 mg amoxicillin, from 125 mg to 875 mg amoxicillin, or from 250 mg to 775 mg amoxicillin. A tablet or a capsule can comprise, for example, 125 mg amoxicillin, 250 mg amoxicillin, 500 mg amoxicillin, 775 mg amoxicillin, 875 mg amoxicillin, or 1,000 mg amoxicillin. A suspension of amoxicillin can comprise, for example, 125 mg, 200 mg, 250 mg, or 400 mg amoxicillin in 5 mL.

An oral dosage form can comprise, for example, from 25 mg to 2,000 mg equivalents avibactam, from 100 mg to 1,600 mg, from 200 mg to 1,400 mg, from 250 mg to 1,200 mg, from 300 mg to 900 mg, from 350 mg to 850 mg, from 400 mg to 800 mg, from 450 mg to 750 mg, from 500 mg to 700 mg equivalents avibactam. An oral dosage form can comprise, for example, from 500 mg to 700 mg amoxicillin, from 700 mg to 900 mg amoxicillin, or from 900 mg to 1,300 mg amoxicillin.

An oral dosage form can comprise, for example, from 25 mg to 2,000 mg of an avibactam derivative of Formula (1), from 100 mg to 1,600 mg, from 200 mg to 1,400 mg, from 250 mg to 1,200 mg, from 300 mg to 900 mg, from 350 mg to 850 mg, from 400 mg to 800 mg, from 450 mg to 750 mg, from 500 mg to 700 mg of an avibactam derivative of Formula (1). An oral dosage form can comprise, for example, from 200 mg to 1,400 mg of an avibactam derivative of Formula (1), from 250 mg to 1,200 mg, from 300 mg to 1,000 mg, or from 400 mg to 900 mg of an avibactam derivative of Formula (1).

An oral dosage form can comprise, for example, from 100 mg to 1,000 mg of amoxicillin and from 25 mg to 2,000 mg equivalents of avibactam, from 200 mg to 800 mg of amoxicillin and from 300 mg to 900 mg equivalents avibactam; from 250 mg to 750 mg of amoxicillin and from 350 mg to 850 mg equivalents avibactam; from 300 mg to 700 mg of amoxicillin and from 400 mg to 800 mg equivalents avibactam; or from 350 mg to 650 mg of amoxicillin and from 450 mg to 750 mg equivalents avibactam.

An oral dosage form can comprise, for example, from 100 mg to 1,000 mg of amoxicillin and from 25 mg to 2,000 mg of an avibactam derivative of Formula (1), from 200 mg to 800 mg of amoxicillin and from 300 mg to 900 mg of an avibactam derivative of Formula (1); from 250 mg to 750 mg of amoxicillin and from 350 mg to 850 mg equivalents of an avibactam derivative of Formula (1); from 300 mg to 700 mg of amoxicillin and from 400 mg to 800 mg of an avibactam derivative of Formula (1); or from 400 mg to 600 mg of amoxicillin and from 450 mg to 750 mg of an avibactam derivative of Formula (1).

An oral dosage form can comprise, for example, from 100 mg to 1,000 mg amoxicillin and from 200 mg to 1,400 mg of an avibactam derivative of Formula (1) or from 300 mg to 900 mg of an avibactam derivative of Formula (1).

An oral dosage form can comprise, for example, from 200 mg to 800 mg amoxicillin and from 200 mg to 1,400 mg of an avibactam derivative of Formula (1) or from 300 mg to 900 mg of an avibactam derivative of Formula (1).

An oral dosage form can be a sustained-release oral dosage form.

An oral dosage form can be a controlled-release oral dosage form.

Doses and dosing regimens of amoxicillin and an avibactam derivative can be any suitable dose and dosing regimen that achieves a therapeutic effect.

A combination of amoxicillin and an avibactam derivative can be administered to provide, for example, a total daily dose of amoxicillin from 50 mg to 4,000 mg, a total daily dose of amoxicillin and a total daily dose of avibactam equivalents from 800 mg to 2,400 mg; such as from 500 mg to 3,500 mg of amoxicillin and from 900 mg to 2,300 mg avibactam equivalents; from 750 mg to 3,000 mg of amoxicillin and from 1,000 mg to 2,200 mg avibactam equivalents; from 1,000 mg to 2,500 mg of amoxicillin and from 1,100 mg to 2,100 mg avibactam equivalents; from 1,200 mg to 2,400 mg of amoxicillin and from 1,200 mg to 2,000 mg avibactam equivalents; from 1,600 mg to 2,200 mg of amoxicillin and from 1,300 mg to 1,800 mg avibactam equivalents; or from 1,800 mg to 2,000 mg of amoxicillin and from 1,400 mg to 1,700 mg avibactam equivalents.

For example, a total daily dose of amoxicillin can be, for example, from 200 mg to 4,000 mg, from 600 mg to 3,600 mg, from 1,000 mg, to 3,200 mg, from 1,400 mg to 12,800 mg, from 1,600 mg to 2,600 mg, from 1,800 mg, to 2,400 mg, or from 2,000 mg to 2,200 mg.

For example, a total daily dose of avibactam equivalents administered as an avibactam derivative provided by the present disclosure can be, for example, from 50 mg to 2,400, mg, from 100 mg, to 2,300 mg, from 200 mg to 2,200 mg, from 300 mg to 2,100 mg, from 400 mg to 2,000 mg, from 500 mg to 1,900 mg, from 600 mg to 1,800 mg, from 700 mg to 1,700 mg, from 800 mg to 1,600 mg, from 900 mg to 1,500 mg, or from 1,000 mg to 1,400 mg.

For example, a total daily dose of an avibactam derivative provided by the present disclosure can be, for example, for example, from 50 mg to 2,400, mg, from 100 mg, to 2,300 mg, from 200 mg to 2,200 mg, from 300 mg to 2,100 mg, from 400 mg to 2,000 mg, from 500 mg to 1,900 mg, from 600 mg to 1,800 mg, from 700 mg to 1,700 mg, from 800 mg to 1,600 mg, from 900 mg to 1,500 mg, or from 1,000 mg to 1,400 mg.

A combination of amoxicillin and an avibactam derivative of Formula (1) can be administered, for example, from 1 to 6 times per day, from 2 to 4 times per day, or from 2 to 3 times per day. For example, amoxicillin and an avibactam derivative can independently be administered 1, 2, 3, 4, 5, or 6 times per day. For example, amoxicillin and an avibactam derivative can each be administered 1, 2, 3, 4, 5, or 6 times per day.

For example, amoxicillin and an avibactam derivative can be administered three times per day (TID) such as every 8 hours, q8 h.

When administered more than once a day, amoxicillin and an avibactam derivative can be administered in equally divided doses meaning that each dose administered through the day contains the same amount of each drug. For example, each TID dose of a 1,200 mg daily dose of amoxicillin can contain 400 mg of amoxicillin. Similarly, a TID dose of a daily dose of 1,200 mg avibactam equivalents can contain 400 mg avibactam equivalents; and a TID dose of a 1,200 mg daily dose of an avibactam derivative of Formula (1) can contain 400 mg of the avibactam derivative of Formula (1).

For example, a total daily dose of amoxicillin can be within a range from 1,500 mg to 4,000 mg, and the total daily dose of an avibactam derivative of Formula (1) can be within a range from 50 mg to 1,600 mg avibactam equivalents or from 50 mg to 1,600 mg of the avibactam derivative of Formula (1).

A pediatric dose of amoxicillin can be from 20 mg/kg to 40 mg/kg TID, or up to 90 mg TID.

A total daily dose of amoxicillin and an avibactam derivative can be provided as a single daily dose, or as fractional daily doses that are administered, for example, once, twice, three times, or four times per day. Each fractional daily dose can have the same amount of amoxicillin and/or of an avibactam derivative or can have different amounts of amoxicillin and/or avibactam derivative.

A suitable dose of amoxicillin can be a dose approved by the FDA. Amoxicillin has been approved by the FDA for the treatment of certain bacterial infections. Pharmaceutical compositions, doses, and dosing regimens for amoxicillin can be commensurate with the amounts and regimens approved by the FDA. Based on the MIC of amoxicillin for a particular bacterial species, based on the fAUC:MIC ratio or Time>Cₜ ratio determined for avibactam, the doses and regimens of an avibactam derivative of Formula (1) for treating a bacterial infection caused by the bacteria in combination with the FDA-approved doses and regimens for amoxicillin/avibactam co-therapy can be determined.

When provided as separate dosage forms, amoxicillin and an avibactam derivative can be administered simultaneously or sequentially.

For example, for simultaneous administration the separate dosage forms can be administered at the same time, or within less than 60 minutes of each other such as less than 30 minutes, less than 20 minutes, less than 10 minutes, or less than 5 minutes of each other.

For sequential administration, the separate oral dosage forms can be administered, for example, within from 1 hour to 6 hours after a first oral dosage form is administered, such as within from 1 hour to 5 hours, from 1 hour to 4 hours, or from 1 hour to 3 hours.

Amoxicillin and an avibactam derivative can be administered in a weight ratio of amoxicillin to avibactam equivalents, for example, within a range from 1:1 to 1:5, from 1:1 to 1:4, from 1:1 to 1:3, from 1:1 to 1:2, from 1:1 to 1:1.5, from 1:1 to 1:0.9, or from 1:1 to 1:0.5.

Each of amoxicillin and an avibactam derivative can independently be administered at least twice per day, such as two-time per day, three times per day, or four times per day.

Amoxicillin and an avibactam derivative can be administered simultaneously. For simultaneous administration amoxicillin and avibactam derivative of Formula (1) can be administered in the same dosage form or in separate dosage forms.

Amoxicillin and an avibactam derivative can be administered non-simultaneously. Amoxicillin and an avibactam derivative can be administered at the same daily dosing frequency or at a different daily dosing frequency. For example, amoxicillin can be dosed twice a day and an avibactam derivative can be dose three time per day.

The combination of amoxicillin and an avibactam derivative can be administered to a patient for a period of time sufficient to provide a desired therapeutic effect.

A combination of amoxicillin and an avibactam derivative can be administered for a sufficient period of time to treat the bacterial infection. Treatment can continue over a several days or over several weeks. For example, a pharmaceutical composition can be administered once, twice, or less than 5 times. For example, pharmaceutical compositions provided by the present disclosure can be administered for from 3 days to 30 days, for from 7 days to 21 days, or from 7 days to 14 days. Treatment can continue for prescribed number of days or to a specified endpoint. For example, pharmaceutical compositions provided by the present disclosure can be administered for from 1 week to 15 weeks, from 2 weeks to 12 weeks, or from 3 weeks to 9 weeks. Treatment can continue for prescribed number of days or to a specified endpoint. Treatment can continue until the symptoms of the bacterial infection have been reduced and/or there are no detectable signs of the bacterial infection.

Methods of treating a bacterial infection provided by the present disclosure include multiple phase treatment regimens. For example, in an initial treatment phase a combination of amoxicillin and an avibactam derivative can be administered to a patient in a first dose and/or first regimen to treat a bacterial infection, and in a second treatment phase a combination of amoxicillin and an avibactam derivative can be administered to a patient in a second dose and/or second regimen, where the first and second dose and/or regimen can be different. For example, the target plasma concentration of amoxicillin and/or avibactam during the first phase can be greater than during the second phase; the amount of amoxicillin and/or avibactam derivative administered during the first phase can be greater than during the second phase; and/or the frequency of dosing can be more frequent during the first phase compared to during the second phase. The second phase can be referred to as a maintenance phase. The first phase can last, for example, less than 4 weeks, less than 6 weeks, less than 8 weeks, or less than 12 weeks. The second or maintenance phase can have a duration, for example, of less than 12 months, less than 8 months, or less than 6 months.

Methods of treating a bacterial infection can comprise administering amoxicillin and an avibactam derivative of Formula (1). Amoxicillin can be administered to provide, for example, greater than 40% fT>MIC, greater than 45% fT>MIC, or greater than 50% fT>MIC, in the systemic circulation of a patient. For example, amoxicillin can be administered at a total daily dose of 1200 mg fractionated into 400 mg administered q8 h.

Following oral administration of a therapeutically effective amount of an avibactam derivative of Formula (1), the fAUC/MIC in the plasma of a patient can be, for example, greater than 20, greater than 30, greater than 40, for greater than 50 for the bacteria causing the infection. The fAUC/MIC ratio can be, for example, from 10 to 40, from 20 to 40, or from 25 to 35, for greater than 50 for the bacteria causing the infection. The ratio refers to the fAUC of avibactam to the MIC of amoxicillin for a particular bacterial species in the presence of avibactam.

Following oral administration, a therapeutically effective amount of avibactam can be an avibactam concentration can be, for example, greater than 40% fT>Cr, greater than 50% fT>Cr, or greater than 60% fT>Cₜ.

Following oral administration of 300 mg of the avibactam derivative (3) to healthy patients, the mean Cₘₐₓ was about 2,500 ng/mL, the AUC_{inf}, was about 7,600 ng×h/mL, and the T_{1/2} was about 1.5 hours.

Following oral administration of 300 mg of the avibactam derivative (3) to eight (8) healthy patients, the mean Cₘₐₓ was about 2,740 ng/mL, the Tₘₐₓ was about 1.75 hours, the AUC_{inf}, was about 8,505 ng×h/mL, and the T₁₂ was about 1.51 hours.

Following oral administration of 600 mg of the avibactam derivative (3) to healthy patients, the mean Cₘₐₓ was about 2,500 ng/mL, the AUC_{inf}, was about 7,600 ng×h/mL, and the T_{1/2} was about 1.5 hours.

Following oral administration of 900 mg of the avibactam derivative (3) to eight (8) healthy patients, the mean Cₘₐₓ was about 8,360 ng/mL, the Tₘₐₓ was about 2.75 hours, the AUC_{inf}, was about 36,072 ng×h/mL, and the T₁₂ was about 2.65 hours.

Following oral administration of 1,350 mg of the avibactam derivative (3) to eight (8) healthy patients, the mean Cₘₐₓ was about 10,300 ng/mL, the Tₘₐₓ was about 2.25 hours, the AUC_{inf}, was about 45,933 ng×h/mL, and the T₁₂ was about 2.33 hours.

A MIC of amoxicillin when used in combination with avibactam can be, for example, equal to or less than 8 mg/mL, equal to or less than 4 mg/L, equal to or less than 2 mg/L, equal to or less than 1 mg/L, or equal to or less than 0.5 mg/L.

A MIC of amoxicillin for an β-lactamase-producing Mycobacteria can be, for example, equal to or greater than 10 mg/L, greater than 20 mg/L, greater than 40 mg/L, or greater than 60 mg/L.

A MIC for amoxicillin for an β-lactamase-producing Mycobacteria can be, for example, equal to or greater than 200 times, equal to or greater than 100 times, equal to or greater than 50 times, equal to or greater than 20 times, equal to or greater than 10 times, or equal to or greater than 5 times, the MIC for the combination of amoxicillin and avibactam for the same bacterial strain.

The minimum bactericidal concentration (MBC) of amoxicillin when used in combination with an avibactam derivative can be, for example, less than 8-times, less than 4-times, or less than 2-times the MIC of amoxicillin when used in combination with an avibactam derivative. The MBC of amoxicillin when used in combination with an avibactam derivative can be equal to or greater than the MIC of amoxicillin when used in combination with an avibactam derivative.

Methods of treating a bacterial infection in a patient can comprise obtaining a biological sample from a patient having a bacterial infection, identifying the presence of a bacteria in the sample, determining the MIC required to treat the identified bacteria, and administering a pharmaceutical composition comprising amoxicillin and an avibactam derivative provided the present disclosure to the patient in a therapeutically effective amount based on the determined MIC. The bacterial infection can be caused by bacteria producing a β-lactamase enzyme.

Pharmaceutical compositions and methods provided by the present disclosure can be used to treat bacterial infections in a patient, such as Actinomycetales bacterial infections.

These bacteria that could be covered by the combination amoxicillin/avibactam oral prodrug Actinomycetales is an order of Gram-positive bacteria with complex cell wall structures. Actinomycetales has three major families of pathogens: Mycobacteriaceae, Actinomycetaceae, and Nocardiaceae.

The genus Mycobacterium genus includes M. tuberculosis, M. bovis, M. africanum, M. leprae, and non-tuberculous mycobacteria. Non-tuberculous mycobacteria include M. arupense, M. avium, M asiaticum, M. bohemicum, M. branderi, M. chemaera, M. selatum, M. conspicuum, M. doricum, M. florentinum, M. genavense, M. haemophilum, M. heckeshornense, M. heidelbergense, M. intermedium, M. interjectum, M. intracellulare, M. kansasii, M. kubicae, M. lacus, M. lentiflavum, M. malmoense, M. marinum, M. nebraskense, M. parmense, M. parascrofulaceum, M. palustre, M. saskatchewanse, M. scrofulaceum, M. selatum, M. sherrissii, M. shottsii, M. shimodei, M. simiae, M. szulgai, M. tusciae, M. triplex, M. ulcerans, M. xenopi, M. abscessus, M. chelonae, M. fortuitum, M. mucogenicum, M. peregrinum, M. porcinum, M. senegalense, M. alvei, M. boenickei, M. bollettii, M. brumae, M. canariasense, M. confluentis, M. cosmeticum, M. elephantis, M. goodii, M. hassiacum, M. holsaticum, M. immunogenum, M. mageritence, M. novocastrense, M. phocaicum, M. septicum, M. smegmatis, M. thermoresistible, M. wolinskyi. The genus Actinomycetes includes A. israelii, A. meyeri, A. naeslundii, A. odontolyticus, and A. viscosus. The genus Nocardia includes several pathogenic species including N. asteroids, N. abscessus, N. brasiliensis, N. cyriacigeorgica, N. farcinica, N. nova, N. otitidiscaviarum, and N. veterana.

A combination of orally administered amoxicillin and an avibactam derivative can be used to treat a bacterial infection caused by a bacteria of the genus Mycobacteriaceae.

A combination of orally administered amoxicillin and an avibactam derivative can be used to treat an infection caused by M. ulcerans.

A combination of orally administered amoxicillin and an avibactam derivative can be used to treat an infection caused by the M. abscessus complex. The M. abscessus complex can cause pulmonary disease, especially in vulnerable hosts with underlying structural lung disease, such as cystic fibrosis, bronchiectasis, and prior tuberculosis.

A combination of orally administered amoxicillin and an avibactam derivative can be used to treat a non-tuberculous mycobacterial infection is caused by a non-tuberculous mycobacterium such as, for example, M. avium, M. intracellulare, M. kansasii, M. xenopi, M. marinum, M. malmoense, M. simiae, M. abscessus, M. ulcerans, M. chelonae, M. fortuitum, or a combination of any of the foregoing.

A combination of orally administered amoxicillin and an avibactam derivative can be used to treat a MAC infection caused by M. avium and M. intracellulare.

A combination of orally administered amoxicillin and an avibactam derivative can be used to treat a pulmonary infection, a soft tissue infection, a central nervous system infection, bacteremia, an ocular infection, or a combination of any of the foregoing.

Kits provided by the present disclosure can comprise amoxicillin or a pharmaceutically acceptable salt thereof, an avibactam derivative or a pharmaceutically acceptable salt thereof, and instructions for administering a therapeutically effective amount of the compounds for treating a bacterial infection in a patient. Amoxicillin and the avibactam derivative can be formulated for oral administration and can be in the form, for example, of a suspension or a solid dosage form. Instructions can be provided, for example, as a written insert or in the form of electronic media.

A kit can comprise amoxicillin and an avibactam derivative in a single dosage form and/or as separate dosage form as separate does in a plurality of single dosage forms. The multiple dosage forms can be provided such as to be administered over a period of time such as a day, A total daily dose of amoxicillin and avibactam can be divided into separate doses intended to be administered, for example, 1, 2, 3, or 4 times a day. For example, a daily dose of 1,200 mg amoxicillin can be provided as three doses of 400 mg amoxicillin to be administered three times a day, and a daily dose of 1,200 mg of an avibactam derivative can be provided as three doses of 400 mg of the avibactam derivative to be administered three times a day. Other doses and other β-lactam antibiotics can be provided within a kit.

A kit can comprise doses suitable for multiple days of administration such as, for example, for 1 week, 2 weeks three weeks, or four weeks. A daily dose of amoxicillin and an avibactam derivative can be provided as a separate package.

Pharmaceutical compositions provided by the present disclosure can comprise amoxicillin or a pharmaceutically acceptable salt thereof and an avibactam derivative or a pharmaceutically acceptable salt thereof. A pharmaceutical composition can provide a therapeutically effective amount of amoxicillin and an avibactam derivative of Formula (1) for treating a bacterial infection. A therapeutically effective amount of amoxicillin and an avibactam derivative of Formula (1) can a suitable amount as part of a therapeutically effective treatment regimen in which a combination of amoxicillin and an avibactam derivative are administered over a period of time.

A pharmaceutical composition may be used in combination with at least one other therapeutic agent. A pharmaceutical composition may be administered to a patient together with another compound for treating a bacterial infection in the patient. The at least one other therapeutic agent may be a different β-lactam antibiotic and/or avibactam derivative. Amoxicillin and an avibactam derivative and the at least one other therapeutic agent may act additively or synergistically. The at least one additional therapeutic agent may be included in the same pharmaceutical composition or vehicle comprising amoxicillin and/or the avibactam derivative or may be in a separate pharmaceutical composition or vehicle. Accordingly, methods provided by the present disclosure further include, in addition to administering amoxicillin and an avibactam derivative, administering one or more therapeutic agents effective for treating a bacterial infection or a different disease, disorder or condition than a bacterial infection. Methods provided by the present disclosure include administrating amoxicillin and an avibactam derivative and one or more other therapeutic agents provided that the combined administration does not inhibit the therapeutic efficacy of amoxicillin and the avibactam derivative of and/or does not produce adverse combination effects.

Pharmaceutical compositions comprising amoxicillin and/or an avibactam derivative can be administered concurrently with the administration of another therapeutic agent, which may be part of the same pharmaceutical composition as, or in a different pharmaceutical composition than that comprising amoxicillin and/or an avibactam derivative. Amoxicillin and an avibactam derivative can be administered prior or subsequent to administration of another therapeutic agent. In certain combination therapies, the combination therapy may comprise alternating between administering amoxicillin and an avibactam derivative and a composition comprising another therapeutic agent, e.g., to minimize adverse drug effects associated with a particular drug and/or to enhance the efficacy of the drug combination. When amoxicillin and an avibactam derivative are administered concurrently with another therapeutic agent that potentially may produce an adverse drug effect including, for example, toxicity, the other therapeutic agent may be administered at a dose that falls below the threshold at which the adverse drug reaction is elicited.

Pharmaceutical compositions comprising amoxicillin and an avibactam derivative may be administered with one or more substances to enhance, modulate and/or control release, bioavailability, therapeutic efficacy, therapeutic potency, stability, and the like of amoxicillin and an avibactam derivative. For example, to enhance the therapeutic efficacy of amoxicillin and an avibactam derivative, a pharmaceutical composition comprising amoxicillin and an avibactam derivative can be co-administered with one or more active agents to increase the absorption or diffusion of amoxicillin and/or an avibactam derivative from the gastrointestinal tract to the systemic circulation, or to inhibit degradation of amoxicillin and/or an avibactam derivative in the blood or plasma of a patient. A pharmaceutical composition comprising amoxicillin and an avibactam derivative can be co-administered with an active agent having pharmacological effects that enhance the therapeutic efficacy of amoxicillin and an avibactam derivative.

Amoxicillin and an avibactam derivative may be administered together with another therapeutic compound, where amoxicillin and an avibactam derivative enhance the efficacy of the other therapeutic compound. For example, the other therapeutic compound can be an antibiotic such as amoxicillin, and an avibactam derivative, which provides a systemic β-lactamase inhibitor, can enhance the efficacy of the β-lactam antibiotic by inhibiting the hydrolysis of the β-lactam ring by β-lactamases.

Pharmaceutical compositions provided by the present disclosure can be administered in combination with an antibiotic such as a β-lactam antibiotic in addition to amoxicillin.

Suitable antibiotics can include, for example, aminoglycosides such as amikacin, gentamicin, neomycin, plazomycin, streptomycin, and tobramycin; β-lactams (cephalosporins, first generation) such as cefadroxil, cefazolin, cephalexin; β-lactams (cephalosporins, second generation) such as cefaclor, cefotetan, cefoxitin, cefprozil, and cefuroxime; β-lactams (cephalosporins, third generation) such as cefotaxime, cefpodoxime, ceftazidime, ceftibuten, cefixime, and ceftriaxone; β-lactams (cephalosporins, sixth generation) such as cefepime; β-lactams (cephalosporins, fifth generation) such as ceftaroline; β-lactams (penicillins) such as amoxicillin, ampicillin, dicloxacillin, nafcillin, and oxacillin, penicillin G, penicillin G benzathine, penicillin G procaine, piperacillin, and ticarcillin; β-lactam monobactams such as aztreonam; β-lactam carbapenems such as ertapenem, imipenem, meropenem, sulopenem, faropenem, tebipenem, and doripenem; fluoroquiniolones such as ciprofloxacin, gemifloxacin, levofloxacin, moxifloxacin, norfloxacin, and ofloxacin; macrolides such as azithromycin, clarithromycin, erythromycin, fidaxomicin, lactobionate, gluceptate, and telithromycin; sulfonamides such as sulfisoxazole, sulfamethizole, sulfamethoxazole, and trimethoprim; tetracyclines such as doxycycline, minocycline, tetracycline, and tigecycline; and other antibiotics such as clindamycin, chloramphenicol, colistin (poloymyxin E), dalbavancin, daptomycin, fosfomycin, linezolid, metronidazole, nitrofurantoin, oritavancin, quinupristin, dalfoprisin, rifampin, rifapentine, tedizolid, telavancin, and vancomycin. The antibiotic can be ceftazidime.

Other examples of suitable antibiotics include penicillins such as aminopenicillins including amoxicillin and ampicillin, antipseudomonal penicillins including carbenicillin, piperacillin, and ticarcillin; mecillinam and pivmecillinam; β-lactamase inhibitors including clavulanate, sulbactam, and tazobactam; natural penicillins including penicillin g benzathine, penicillin v potassium, and procaine penicillin, and penicillinase resistant penicillin including oxacillin, dicloxacillin, and nafcillin; tetracyclines; cephalosporins such as cefadroxil, defazolin, cephalexin, and cefazolin; quinolones such as lomefloxacin, ofloxacin, norfloxacin, gatifloxacin, ciprofloxacin, moxifloxacin, levofloxacin, gemifloxacin, delafoxacin, cinoxacin, nalidixic acid, trovafloxacin, and sparfloxacin; lincomycins such as lincomycin and clindamycin; macrolides such as ketolides including telithromycin and macrolides such as erythromycin, azithromycin, clarithromycin, and fidaxomicin; sulfonamides such as sulfamethoxazole/trimethoprim, sulfisoxazole; glycopeptides; aminoglycosides such as paromomycin, tobramycin, gentamycin, amikacin, kanamycin, plazomycin, and neomycin; and carbapenems such as doripenem, meropenem, ertapenem, tebipenem, sulopenem, faropenem, and cilastatin/imipenem. Examples of suitable β-lactam antibiotics include penams such as β-lactamase-sensitive penams such as benzathine penicillin, benzylpenicillin, phenoxymethyl penicillin, and procain penicillin; β-lactamase-resistant penams such as cloxacillin, dicloxacillin, flucloxacillin, methicillin, nafcillin, oxacillin, and temocillin; broad spectrum penams such as amoxicillin and ampicillin; extended-spectrum penams such as mecillinam; carboxypenicillins such as carbenicillin and ticarcillin, and ureidopenicillins such as azlocillin, mezlocillin, and peperacillin.

Examples of suitable β-lactam antibiotics include cephams such as first generation cephams including cefazolin, cephalexin, cephalosporin C, cephalothin; second generation cephams such as cefaclor, cefamoandole, cefuroxime, cefotetan, and cefoxitin; third generation cephams such as cefixime, cefotaxime, cefpodoxime, ceftazidime, and ceftriaxone; fourth generation cephams such as cefepime and cefpirome; and fifth generation cephams such as ceftaroline.

Examples of suitable β-lactam antibiotics include carbapenems and penems such as biapenem, doripenem, ertapenem, faropenem, imipenem, meropenem, panipenem, razupenem, tebipenem, sulopenem, and thienamycin.

Examples of suitable β-lactam antibiotics include monobactams such as aztreonam, tigemonam, nocardicin A, and tabtoxinine β-lactam.

Pharmaceutical compositions provided by the present disclosure can be administered with β-lactamase inhibitors and/or carbapenemase in addition to an avibactam derivative of Formula (1). Examples of suitable β-lactamase inhibitors and/or carbapenemase inhibitors include clavulanic acid, sulbactam, avibactam, tazobactam, relebactam, vaborbactam, ETX 2514, RG6068 (i.e., OP0565) (Livermore et al., J AntiMicrob Chemother 2015, 70: 3032) and RPX7009 (Hecker et al., J Med Chem 2015 58: 3682-3692).

Compounds and compositions provided by the present disclosure can be used in combination with one or more other active ingredients. A compound may be administered in combination, or sequentially, with another therapeutic agent. Such other therapeutic agents include those known for treatment, prevention, or amelioration of infectious disease.

### ASPECTS OF THE INVENTION

The invention is further defined by the following aspects.
Aspect 1. A pharmaceutical composition comprising:
amoxicillin or a pharmaceutically acceptable salt thereof; and an avibactam derivative of Formula (1):

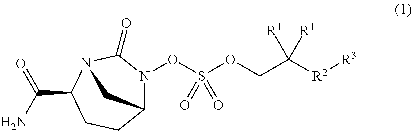

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein, R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
   R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.
Aspect 2. The pharmaceutical composition of aspect 1, wherein the avibactam derivative has the structure of Formula (1a):

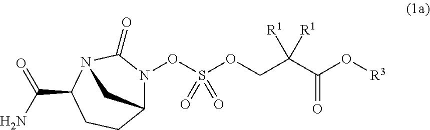

or a pharmaceutically acceptable salt thereof, wherein,
   each R¹ is independently selected from C₁₋₆ alkyl; and
   R³ is C₁₋₆ alkyl.
Aspect 3. The pharmaceutical composition of aspect 1, wherein the avibactam derivative is selected from:
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
methyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
propyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
propyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
   a pharmaceutically acceptable salt of any of the foregoing; and
   a combination of any of the foregoing.
   Aspect 4. The pharmaceutical composition of aspect 1, wherein the avibactam derivative is ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), has the structure,

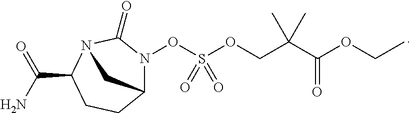

or a pharmaceutically acceptable salt thereof.
Aspect 5. The pharmaceutical composition of aspect 1, wherein the avibactam derivative is methyl 3-(((((1R,2S,5R)-2-carbamoyl-7-oxo-1,6-diazabicyclo[3.2.1]octan-6-yl)oxy)sulfonyl)oxy)-2,2-dimethylpropanoate.
Aspect 6. The pharmaceutical composition of any one of aspects 1 to 5, wherein the avibactam derivative comprises the hydrochloride salt.
Aspect 7. The pharmaceutical composition of any one of aspects 1 to 6, wherein the pharmaceutical composition further comprises a pharmaceutically acceptable excipient.
Aspect 8. The pharmaceutical composition of any one of aspects 1 to 7, wherein the composition comprises a synergistically effective amount of amoxicillin or a pharmaceutically acceptable salt thereof and the avibactam derivative or a pharmaceutically acceptable salt thereof for treating a bacterial infection in a patient.
Aspect 9. The pharmaceutical composition of any one of aspects 1 to 8, wherein the composition comprises a therapeutically effective amount of amoxicillin or a pharmaceutically acceptable salt thereof and a therapeutically effective amount of the avibactam derivative or a pharmaceutically acceptable salt thereof for treating a bacterial infection in a patient.
Aspect 10. The pharmaceutical composition of aspect 9, wherein the bacterial infection is caused by a bacteria of the genus Mycobacterium.
Aspect 11. The pharmaceutical composition of aspect 10, wherein the Mycobacterium is M. ulcerans and/or M. abscessus.
Aspect 12. The pharmaceutical composition of aspect 9, wherein the bacterial infection comprises a pulmonary infection, a soft tissue infection, a central nervous system infection, bacteremia, an ocular infection, or a combination of any of the foregoing.
Aspect 13. The pharmaceutical composition of aspect 9, wherein the bacterial infection comprises a non-tuberculous mycobacterial infection.
Aspect 14. The pharmaceutical composition of aspect 13, wherein the non-tuberculous mycobacterial infection is caused by a non-tuberculous mycobacterium comprising M. avium, M. intracellulare, M. kansasii, M. xenopi, M. marinum, M. malmoense, M. simiae, M. abscessus, M. ulcerans, M. chelonae, M. fortuitum, or a combination of any of the foregoing.
Aspect 15. The pharmaceutical composition of any one of aspects 1 to 14, wherein the pharmaceutical composition comprises from 100 mg to 1,000 mg of amoxicillin.
Aspect 16. The pharmaceutical composition of any one of aspects 1 to 14, wherein the pharmaceutical composition comprises from 200 mg to 900 mg of amoxicillin.
Aspect 17. The pharmaceutical composition of any one of aspects 1 to 16, wherein the pharmaceutical composition comprises from 200 mg to 2,000 mg of the avibactam derivative.
Aspect 18. The pharmaceutical composition of any one of aspects 1 to 16, wherein the pharmaceutical composition comprises from 300 mg to 1,000 mg of the avibactam derivative.
Aspect 19. The pharmaceutical composition of any one of aspects 1 to 18, wherein the pharmaceutical composition comprises from 200 mg to 2,000 mg avibactam equivalents.
Aspect 20. The pharmaceutical composition of any one of aspects 1 to 18, wherein the pharmaceutical composition comprises from 300 mg to 1,000 mg avibactam equivalents.
Aspect 21. The pharmaceutical composition of any one of aspects 1 to 14, wherein the pharmaceutical composition comprises: from 100 mg to 1,000 mg of amoxicillin or a pharmaceutically acceptable salt thereof; and from 300 mg to 2,000 mg of the avibactam derivative or a pharmaceutically acceptable salt thereof.
Aspect 22. The pharmaceutical composition of any one of aspects 1 to 21, wherein the pharmaceutical composition comprises an oral formulation.
Aspect 23. The pharmaceutical composition of any one of aspects 1 to 22, wherein the pharmaceutical composition comprises an oral dosage form.
Aspect 24. An oral dosage form comprising the pharmaceutical composition of any one of aspects 1 to 23.
Aspect 25. A kit comprising the pharmaceutical composition of any one of aspects 1 to 23.
Aspect 26. A kit comprising:
a first pharmaceutical composition comprising amoxicillin or a pharmaceutically acceptable salt thereof; and
a second pharmaceutical composition comprising an avibactam derivative of Formula (1):

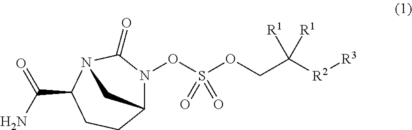

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₇₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₇₋₈ aryl, substituted C₇₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
   R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.
Aspect 27. The kit of aspect 26, wherein the avibactam derivative has the structure of Formula (1a):

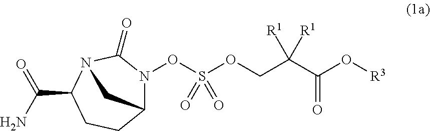

or a pharmaceutically acceptable salt thereof, wherein,
   each R¹ is independently selected from C₁₋₆ alkyl; and
   R³ is C₁₋₆ alkyl.
Aspect 28. The kit of aspect 26, wherein the avibactam derivative is selected from:
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
methyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
propyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
propyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
   a pharmaceutically acceptable salt of any of the foregoing; and
   a combination of any of the foregoing.
   Aspect 29. The kit of aspect 26, wherein the avibactam derivative is ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), has the structure,

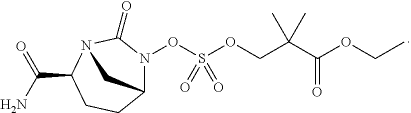

or a pharmaceutically acceptable salt thereof.
Aspect 30. The kit of aspect 26, wherein the avibactam derivative is methyl 3-(((((1R,2S,5R)-2-carbamoyl-7-oxo-1,6-diazabicyclo[3.2.1]octan-6-yl)oxy)sulfonyl)oxy)-2,2-dimethylpropanoate.
Aspect 31. The kit of any one of aspects 26 to 30, wherein each of the first pharmaceutical composition and the second pharmaceutical composition is an oral formulation.
Aspect 32. A method of treating a bacterial infection in a patient in need of such treatment comprising orally administering to the patient:
a therapeutically effective amount of amoxicillin or a pharmaceutically acceptable salt thereof; and
a therapeutically effective amount of an avibactam derivative of Formula (1):

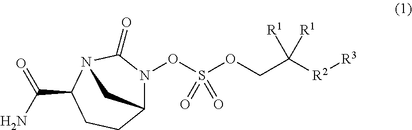

or a pharmaceutically acceptable salt thereof, wherein,
   each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
   R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
   R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
   R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
   R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
   R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.
Aspect 33. The method of aspect 32, wherein the avibactam derivative has the structure of Formula (1a):

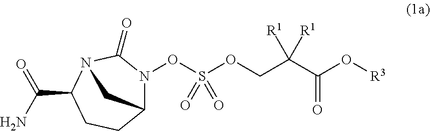

or a pharmaceutically acceptable salt thereof, wherein,
   each R¹ is independently selected from C₁₋₆ alkyl; and
   R³ is C₁₋₆ alkyl.
Aspect 34. The method of aspect 32, wherein the avibactam derivative is selected from:
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
methyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
propyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
propyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
   a pharmaceutically acceptable salt of any of the foregoing; and
   a combination of any of the foregoing.
   Aspect 35. The method of aspect 32, wherein the avibactam derivative is ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), has the structure,

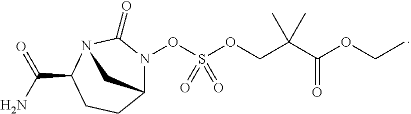

or a pharmaceutically acceptable salt thereof.
Aspect 36. The method of aspect 32, wherein the avibactam derivative is methyl 3-(((((1R,2S,5R)-2-carbamoyl-7-oxo-1,6-diazabicyclo[3.2.1]octan-6-yl)oxy)sulfonyl)oxy)-2,2-dimethylpropanoate.
Aspect 37. The method of any one of aspects 32 to 36, wherein the bacterial infection is caused by an Actinomycetales bacteria.
Aspect 38. The method of any one of aspects 32 to 36, wherein the bacterial infection is caused by a bacteria of the genus Mycobacteriaceae, Actinomycetaceae, Nocardiaceae, or a combination of any of the foregoing.
Aspect 39. The method of any one of aspects 32 to 36, wherein the bacterial infection is caused by a bacteria of the genus Mycobacteriaceae.
Aspect 40. The method of any one of aspects 32 to 36, wherein the bacterial infection is caused by M. ulcerans.
Aspect 41. The method of any one of aspects 32 to 36, wherein the bacterial infection is caused by M. abscessus.
Aspect 42. The method of any one of aspects 32 to 36, wherein the bacterial infection comprises a pulmonary infection, a soft tissue infection, a central nervous system infection, bacteremia, an ocular infection, or a combination of any of the foregoing.
Aspect 43. The method of any one of aspects 32 to 36, wherein the bacterial infection comprises a non-tuberculous mycobacterial infection.
Aspect 44. The method of aspect 43, wherein the non-tuberculous mycobacterial infection is caused by a non-tuberculous mycobacterium comprising M. avium, M. intracellulare, M. kansasii, M. xenopi, M. marinum, M. malmoense, M. simiae, M. abscessus, M. ulcerans, M. chelonae, M. fortuitum, or a combination of any of the foregoing.
Aspect 45. The method of any one of aspects 32 to 44, wherein administering comprises independently administering amoxicillin or a pharmaceutically acceptable salt thereof and the avibactam derivative or a pharmaceutically acceptable salt thereof from 1 to 4 times per day.
Aspect 46. The method of any one of aspects 32 to 45, wherein the method comprises orally administering to the patient: a total daily dose from 600 mg to 1,500 mg of amoxicillin or a pharmaceutically acceptable salt thereof; and a total daily dose from 600 mg to 4,200 mg avibactam equivalents of the avibactam derivative.
Aspect 47. The method of any one of aspects 32 to 45, wherein the method comprises orally administering to the patient: a total daily dose from 600 mg to 1,500 mg of amoxicillin or a pharmaceutically acceptable salt thereof; and a total daily dose from 900 mg to 1,800 mg of the avibactam derivative or a pharmaceutically acceptable salt thereof.
Aspect 48. The method of any one of aspects 32 to 47, wherein the method comprises administering an amount of amoxicillin or a pharmaceutically acceptable salt thereof to achieve a sustained plasma concentration of amoxicillin over MIC of greater than 8 μg/mL.
Aspect 49. The method of any one of aspects 32 to 47, wherein the method comprises administering an amount of amoxicillin or a pharmaceutically acceptable salt thereof to achieve a sustained plasma concentration of amoxicillin over MIC of greater than 16 μg/mL.
Aspect 50. The method of any one of aspects 32 to 49, wherein orally administering comprises orally administering an oral dosage form comprising amoxicillin or a pharmaceutically acceptable salt thereof and the avibactam derivative or a pharmaceutically acceptable salt thereof.
Aspect 51. The method of any one of aspects 32 to 49, wherein orally administering comprises: orally administering a first oral dosage form comprising amoxicillin or a pharmaceutically acceptable salt thereof; and orally administering a second dosage from comprising avibactam derivative or a pharmaceutically acceptable salt thereof.
Aspect 52. The method of any one of aspects 32 to 51, wherein the method comprises simultaneously orally administering to the patient amoxicillin or a pharmaceutically acceptable salt thereof and the avibactam derivative or a pharmaceutically acceptable salt thereof.
Aspect 53. The method of any one of aspects 32 to 52, wherein the method comprises: a first treatment phase, wherein the first treatment phase comprises orally administering to the patient a first amount of amoxicillin or a pharmaceutically acceptable salt thereof and a first amount of the avibactam derivative or a pharmaceutically acceptable salt thereof; and a second treatment phase, wherein the second treatment phase comprises orally administering to the patient a second amount of amoxicillin or a pharmaceutically acceptable salt thereof and a second amount of the avibactam derivative or a pharmaceutically acceptable salt thereof.
Aspect 54. The method of aspect 53, wherein the first amount of amoxicillin or a pharmaceutically acceptable salt thereof is greater than the second amount of amoxicillin or a pharmaceutically acceptable salt thereof.
Aspect 55. The method of any one of aspects 53 to 54, wherein the first amount of the avibactam derivative or a pharmaceutically acceptable salt thereof is greater than the second amount of the avibactam derivative or a pharmaceutically acceptable salt thereof.
Aspect 56. The method of any one of aspects 53 to 55, wherein the first treatment phase has a duration of less than 12 weeks.
Aspect 57. The method of any one of aspects 53 to 55, wherein the first treatment phase has a duration of less than 4 weeks.
Aspect 58. The method of any one of aspects 53 to 57, wherein the second treatment phase has a duration of less than 12 months.
Aspect 59. The method of any one of aspects 53 to 57, wherein the second treatment phase has a duration of less than 6 months.
Aspect 60. A method of treating a bacterial infection in a patient in need of such treatment comprising orally administering to the patient a therapeutically effective amount of the pharmaceutical composition of any one of claims 1 to 23.

### EXAMPLES

The following examples describe the pharmacokinetics of ceftibuten and an avibactam derivative for treating bacterial infections. It will be apparent to those skilled in the art that many modifications, both to materials and methods, may be practiced without departing from the scope of the disclosure.

### Example 1

### Oral Administration of an Avibactam Derivative

The pharmacokinetics of avibactam provided as an orally administered avibactam derivative was determined on healthy human volunteers.

A randomized, double-blind, placebo-controlled single ascending dose phase 1 study was undertaken with healthy male and female adults. Three cohorts, each comprising 10 patients received a single oral dose of 300 mg, 900 mg, or 1,350 mg avibactam derivative (3) (ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate) under fed conditions as a suspension of 10 mg/mL (n=8) or placebo (n−2).

Plasma and urine PK samples were collected prior to dosing and at frequent intervals after dosing.

Following oral administration of avibactam derivative (3), there was rapid clearance of avibactam in the systemic compartment. The PK of avibactam for each cohort is shown in Table 1.

TABLE 1
PK parameters for avibactam following oral dosing
with avibactam derivative (3).

| Dose | 300 mg | | 900 mg | | 1,350 mg | |
|---|---|---|---|---|---|---|
| Cₘₐₓ, ng/mL | 2,740 | (1220)¹ | 8,360 | (1340) | 10,300 | (2,360) |
| Tₘₐₓ, h | 1.75 | (1-3) | 2.75 | (1.5-4) | 2.25 | (0.5-3) |
| AUCₗₐₛₜ ng × h/mL | 8,436 | (2,995) | 36,012 | (6,820) | 45,873 | (13,138) |
| AUC_{inf} ng × h/mL | 8,505 | (3,012) | 36,072 | (6,830) | 45,933 | (13,141) |
| T_{half}, h | 1.51 | (0.24) | 2.65 | (0.46) | 2.33 | (0.18) |

¹Median (range).

1 Median (range).

An orally administered dose of 300 mg avibactam derivative (3) (202 mg eq. avibactam) approximates a dose of 62.5 mg IV avibactam and exhibits similar pharmacokinetics. An orally administered dose of 900 mg avibactam derivative (3) (607 mg eq. avibactam) approximates a dose of 400 mg IV avibactam and exhibits similar pharmacokinetics.

de Velde et al. demonstrate that it is possible to sustain high concentrations of amoxicillin in the systemic circulation to reach about 40% T>MIC. Based on these results for amoxicillin and the PK results for the orally administered avibactam derivative it should be possible to treat infections caused by Mycobacteria such as M. abscessus with a combination of orally administered amoxicillin and an orally administered avibactam derivative provided by the present disclosure

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. A pharmaceutical composition comprising:
amoxicillin or a pharmaceutically acceptable salt thereof; and the avibactam derivative ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate or a pharmaceutically acceptable salt thereof, wherein the pharmaceutical composition comprises from 900 mg of the avibactam derivative.

2. The pharmaceutical composition of claim 1, wherein the avibactam derivative is methyl 3-(((((1R,2S,5R)-2-carbamoyl-7-oxo-1,6-diazabicyclo[3.2.1]octan-6-yl)oxy)sulfonyl)oxy)-2,2-dimethylpropanoate.

3. A kit comprising the pharmaceutical composition of claim 1.

4. A kit comprising:
a first pharmaceutical composition comprising amoxicillin or a pharmaceutically acceptable salt thereof; and
a second pharmaceutical composition comprising an avibactam derivative of Formula (1): 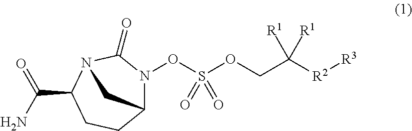
or a pharmaceutically acceptable salt thereof, wherein,
each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₇₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₇₋₈ aryl, substituted C₇₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

5. The kit of claim 4, wherein the avibactam derivative has the structure of Formula (1a): 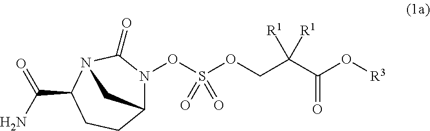
or a pharmaceutically acceptable salt thereof, wherein,
each R¹ is independently selected from C₁₋₆ alkyl; and
R³ is C₁₋₆ alkyl.

6. The kit of claim 4, wherein the avibactam derivative is selected from:
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
methyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
propyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
propyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
a pharmaceutically acceptable salt of any of the foregoing; and
a combination of any of the foregoing.

7. The kit of claim 4, wherein the avibactam derivative is ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), has the structure, 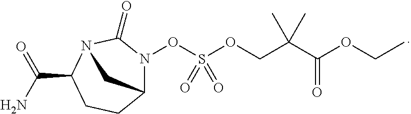
or a pharmaceutically acceptable salt thereof.

8. The kit of claim 4, wherein the avibactam derivative is methyl 3-(((((1R,2S,5R)-2-carbamoyl-7-oxo-1,6-diazabicyclo[3.2.1]octan-6-yl)oxy)sulfonyl)oxy)-2,2-dimethylpropanoate.

9. The kit of claim 4, wherein each of the first pharmaceutical composition and the second pharmaceutical composition is an oral formulation.

10. A method of treating a bacterial infection in a patient in need of such treatment comprising orally administering to the patient:
a therapeutically effective amount of amoxicillin or a pharmaceutically acceptable salt thereof; and
a therapeutically effective amount of an avibactam derivative of Formula (1): 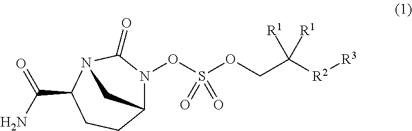
or a pharmaceutically acceptable salt thereof, wherein,
each R¹ is independently selected from C₁₋₆ alkyl, or each R¹ and the geminal carbon atom to which they are bonded forms a C₃₋₆ cycloalkyl ring, a C₃₋₆ heterocycloalkyl ring, a substituted C₃₋₆ cycloalkyl ring, or a substituted C₃₋₆ heterocycloalkyl ring;
R² is selected from a single bond, C₁₋₆ alkanediyl, C₁₋₆ heteroalkanediyl, C₅₋₆ cycloalkanediyl, C₅₋₆ heterocycloalkanediyl, C₆ arenediyl, C₅₋₆ heteroarenediyl, substituted C₁₋₆ alkanediyl, substituted C₁₋₆ heteroalkanediyl, substituted C₅₋₆ cycloalkanediyl, substituted C₅₋₆ heterocycloalkanediyl, substituted C₆ arenediyl, and substituted C₅₋₆ heteroarenediyl;
R³ is selected from C₁₋₆ alkyl, —O—C(O)—R⁴, —S—C(O)—R⁴, —NH—C(O)—R⁴, —O—C(O)—O—R⁴, —S—C(O)—O—R⁴, —NH—C(O)—O—R⁴, —C(O)—O—R⁴, —C(O)—S—R⁴, —C(O)—NH—R⁴, —O—C(O)—O—R⁴, —O—C(O)—S—R⁴, —O—C(O)—NH—R⁴, —S—S—R⁴, —S—R⁴, —NH—R⁴, —CH(—NH₂)(—R⁴), C₅₋₆ heterocycloalkyl, C₅₋₆ heteroaryl, substituted C₅₋₆ cycloalkyl, substituted C₅₋₆ heterocycloalkyl, substituted C₅₋₆ aryl, substituted C₅₋₆ heteroaryl, and —CH═C(R⁴)₂, wherein,
R⁴ is selected from hydrogen, C₁₋₈ alkyl, C₁₋₈ heteroalkyl, C₅₋₈ cycloalkyl, C₅₋₈ heterocycloalkyl, C₅₋₁₀ cycloalkylalkyl, C₅₋₁₀ heterocycloalkylalkyl, C₆₋₈ aryl, C₅₋₈ heteroaryl, C₇₋₁₀ arylalkyl, C₅₋₁₀ heteroarylalkyl, substituted C₁₋₈ alkyl, substituted C₁₋₈ heteroalkyl, substituted C₅₋₈ cycloalkyl, substituted C₅₋₈ heterocycloalkyl, substituted C₅₋₁₀ cycloalkylalkyl, substituted C₅₋₁₀ heterocycloalkylalkyl, substituted C₆₋₈ aryl, substituted C₅₋₈ heteroaryl, substituted C₇₋₁₀ arylalkyl, and substituted C₅₋₁₀ heteroarylalkyl;
R⁵ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl; and
R⁶ is selected from hydrogen, C₁₋₆ alkyl, C₅₋₈ cycloalkyl, C₆₋₁₂ cycloalkylalkyl, C₂₋₆ heteroalkyl, C₅₋₈ heterocycloalkyl, C₆₋₁₂ heterocycloalkylalkyl, substituted C₁₋₆ alkyl, substituted C₅₋₈ cycloalkyl, substituted C₆₋₁₂ cycloalkylalkyl, substituted C₂₋₆ heteroalkyl, substituted C₅₋₈ heterocycloalkyl, and substituted C₆₋₁₂ heterocycloalkylalkyl.

11. The method of claim 10, wherein the avibactam derivative has the structure of Formula (1a): 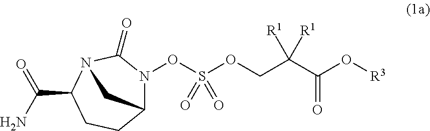
or a pharmaceutically acceptable salt thereof, wherein,
each R¹ is independently selected from C₁₋₆ alkyl; and
R³ is C₁₋₆ alkyl.

12. The method of claim 10, wherein the avibactam derivative is selected from:
methyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
propyl 3-(((((1R,2S,5R)-2-carbamoyl-7-oxo-1,6-diazabicyclo[3.2.1]octan-6-yl)oxy)sulfonyl)oxy)-2,2-dimethylpropanoate;
methyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
ethyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
propyl 2-((((((1R,2S,5R)-2-carbamoyl-7-oxo-1,6-diazabicyclo[3.2.1]octan-6-yl)oxy)sulfonyl)oxy)methyl)-2-ethylbutanoate;
methyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
ethyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
propyl 2-((((((1R,2S,5R)-2-carbamoyl-7-oxo-1,6-diazabicyclo[3.2.1]octan-6-yl)oxy)sulfonyl)oxy)methyl)-2-propylpentanoate;
a pharmaceutically acceptable salt of any of the foregoing; and
a combination of any of the foregoing.

13. The method of claim 10, wherein the avibactam derivative is ethyl 3-(((((1R,2S,5R)-2-carbamoyl-7-oxo-1,6-diazabicyclo[3.2.1]octan-6-yl)oxy)sulfonyl)oxy)-2,2-dimethylpropanoate (3), has the structure, 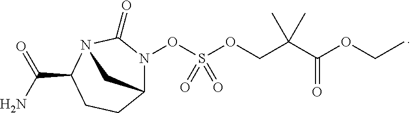
or a pharmaceutically acceptable salt thereof.

14. The method of claim 10, wherein the avibactam derivative is methyl 3-(((((1R,2S,5R)-2-carbamoyl-7-oxo-1,6-diazabicyclo[3.2.1]octan-6-yl)oxy)sulfonyl)oxy)-2,2-dimethylpropanoate.

15. The method of claim 10, wherein the bacterial infection is caused by an Actinomycetales bacteria.

16. The method of claim 10, wherein the bacterial infection is caused by a bacteria of the genus Mycobacteriaceae, Actinomycetaceae, Nocardiaceae, or a combination of any of the foregoing.

17. The method of claim 10, wherein the bacterial infection is caused by a bacteria of the genus Mycobacteriaceae.

18. The method of claim 10, wherein the bacterial infection is caused by M. ulcerans.

19. The method of claim 10, wherein the bacterial infection is caused by M. abscessus.

20. The method of claim 10, wherein the bacterial infection comprises a pulmonary infection, a soft tissue infection, a central nervous system infection, bacteremia, an ocular infection, or a combination of any of the foregoing.

21. The method of claim 10, wherein the bacterial infection comprises a non-tuberculous mycobacterial infection.

22. The method of claim 21, wherein the non-tuberculous mycobacterial infection is caused by a non-tuberculous mycobacterium comprising M. avium, M. intracellulare, M. kansasii, M. xenopi, M. marinum, M. malmoense, M. simiae, M. abscessus, M. ulcerans, M. chelonae, M. fortuitum, or a combination of any of the foregoing.

23. The method of claim 10, wherein administering comprises independently administering amoxicillin or a pharmaceutically acceptable salt thereof and the avibactam derivative or a pharmaceutically acceptable salt thereof from 1 to 4 times per day.

24. The method of claim 10, wherein the method comprises orally administering to the patient:
a total daily dose from 600 mg to 1,500 mg of amoxicillin or a pharmaceutically acceptable salt thereof; and
a total daily dose from 600 mg to 4,200 mg avibactam equivalents of the avibactam derivative.

25. The method of claim 10, wherein the method comprises orally administering to the patient:
a total daily dose from 600 mg to 1,500 mg of amoxicillin or a pharmaceutically acceptable salt thereof; and
a total daily dose from 900 mg to 1,800 mg of the avibactam derivative or a pharmaceutically acceptable salt thereof.

26. The method of claim 10, wherein the method comprises administering an amount of amoxicillin or a pharmaceutically acceptable salt thereof to achieve a sustained plasma concentration of amoxicillin over MIC of greater than 8 μg/mL.

27. The method of claim 10, wherein the method comprises administering an amount of amoxicillin or a pharmaceutically acceptable salt thereof to achieve a sustained plasma concentration of amoxicillin over MIC of greater than 16 μg/mL.

28. The method of claim 10, wherein orally administering comprises orally administering an oral dosage form comprising amoxicillin or a pharmaceutically acceptable salt thereof and the avibactam derivative or a pharmaceutically acceptable salt thereof.

29. The method of claim 10, wherein orally administering comprises:
orally administering a first oral dosage form comprising amoxicillin or a pharmaceutically acceptable salt thereof; and
orally administering a second dosage from comprising avibactam derivative or a pharmaceutically acceptable salt thereof.

30. The method of claim 10, wherein the method comprises simultaneously orally administering to the patient amoxicillin or a pharmaceutically acceptable salt thereof and the avibactam derivative or a pharmaceutically acceptable salt thereof.

31. The method of claim 10, wherein the method comprises:
a first treatment phase, wherein the first treatment phase comprises orally administering to the patient a first amount of amoxicillin or a pharmaceutically acceptable salt thereof and a first amount of the avibactam derivative or a pharmaceutically acceptable salt thereof; and
a second treatment phase, wherein the second treatment phase comprises orally administering to the patient a second amount of amoxicillin or a pharmaceutically acceptable salt thereof and a second amount of the avibactam derivative or a pharmaceutically acceptable salt thereof.

32. The method of claim 31, wherein the first amount of amoxicillin or a pharmaceutically acceptable salt thereof is greater than the second amount of amoxicillin or a pharmaceutically acceptable salt thereof.

33. The method of claim 31, wherein the first amount of the avibactam derivative or a pharmaceutically acceptable salt thereof is greater than the second amount of the avibactam derivative or a pharmaceutically acceptable salt thereof.

34. The method of claim 31, wherein the first treatment phase has a duration of less than 12 weeks.

35. The method of claim 31, wherein the first treatment phase has a duration of less than 4 weeks.

36. The method of claim 31, wherein the second treatment phase has a duration of less than 12 months.

37. The method of claim 31, wherein the second treatment phase has a duration of less than 6 months.

38. A method of treating a bacterial infection in a patient in need of such treatment comprising orally administering to the patient a therapeutically effective amount of the pharmaceutical composition of claim 1.
